(19) United States
(12) Patent Application Publication
(10) Pub. No.: US 2026/0237185 A1
(43) Pub. Date: Aug. 13, 2026

(54) MACHINE LEARNING-BASED OPTORETINOGRAPHY USING PHASE-SENSITIVE OPTICAL COHERENCE TOMOGRAPHY

(71) Applicants: NANYANG TECHNOLOGICAL UNIVERSITY, Singapore (SG); SINGAPORE HEALTH SERVICES PTE LTD, Singapore (SG)

(72) Inventors: Tong LING, Singapore (SG); Leopold SCHMETTERER, Singapore (SG); Bingyao TAN, Singapore (SG); Huakun LI, Singapore (SG)

(21) Appl. No.: 19/151,256

(22) PCT Filed: Jan. 26, 2024
(86) PCT No.: PCT/SG2024/050050

§ 371 Date: Jul. 25, 2025

(30) Foreign Application Priority Data
Jan. 26, 2023 (SG) 10202300210V

(51) Int. Cl. G06V 10/764; A61B 3/00; A61B 3/10; G06T 5/50; G06T 5/60; G06V 10/762; G06V 10/774

(52) U.S. Cl. CPC G06V 10/764; A61B 3/0025; A61B 3/102; G06T 5/50; G06T 5/60; G06V 10/762; G06V 10/774; G06T 2207/10101; G06T 2207/20081; G06T 2207/20216; G06T 2207/30041; G06V 2201/03

(57) ABSTRACT

A method of training a machine learning model for classifying a temporal phase signal obtained from OCT images produced for optoretinography is provided. The method includes: obtaining a series of OCT images produced by a phase-sensitive OCT system for optoretinography with respect to an outer retina, including a stimulus sub-series of OCT images produced in response to a visual stimulus to the outer retina; determining, for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images.

### CROSS-REFERENCE TO RELATED APPLICATION

This application claims the benefit of priority of Singapore Patent Application No. 10202300210V filed on 26 Jan. 2023, the content of which being hereby incorporated by reference in its entirety for all purposes.

### TECHNICAL FIELD

The present invention generally relates to machine learning-based optoretinography (ORG) using phase-sensitive optical coherence tomography (OCT), and more particularly, a method of training a machine learning model for classifying a temporal phase signal obtained from OCT images produced for optoretinography and a system thereof, as well as a method of classifying a temporal phase signal obtained from OCT images produced for optoretinography using the trained machine learning model and a system thereof.

### BACKGROUND

Phototransduction involves changes in concentration of ions and other solutes within photoreceptors and in subretinal space, which affect osmotic pressure and the associated water flow. Ion homeostasis of subretinal space is regulated by retinal pigment epithelium (RPE), a monolayer of cells between the photoreceptor outer segment and the choroid, and a variety of ocular diseases, such as age-related macular degeneration, retinitis pigmentosa and Best's vitelliform macular dystrophy, are associated with dysfunction of epithelial transport of RPE cells. In-vivo assessment of RPE ion transport relies mostly on c-wave in electroretinography (ERG) or electrooculography, which require contact electrodes and provide rather low spatial resolution.

Optoretinography (ORG) is an emerging imaging technology for non-invasive optical probing of retinal physiology in vivo. In particular, optoretinography is an optical imaging modality for non-invasively diagnosing photoreceptor function in vivo. It usually utilizes phase-sensitive optical coherence tomography (OCT) to detect mechanical deformations of retinal cells in response to visual stimuli, for example, the deformation of photoreceptors' outer segments (OS), which is associated with photoisomerization and phototransduction. Accordingly, optoretinography measures the function-associated mechanical deformations of specialized neurons in the retina using OCT. Unlike conventional OCT, where its axial motion sensitivity is constrained by the bandwidth-limited axial resolution (typically >1 μm), axial sensitivity of the phase-sensitive OCT is defined by the signal-to-noise ratio, potentially enabling the detection of nanometer-scale tissue dynamics. For example, optoretinography was recently employed to classify chromatic cone subtypes in human subjects based on their responses to chromatic flashes of various colors, and its clinical potential was demonstrated in assessing the progression of retinitis pigmentosa. Furthermore, considering the similarity in photoreceptors' structure and function across mammals, small animals such as rodents, serve as convenient and cost-effective models for studying the mechanisms of photoreceptor degeneration using optoretinography. In genetically modified animals, for example Gnat1 and Gnat2 mutants and knockouts, optoretinography can provide new insights into the various stages of the phototransduction cascade. In addition, unlike human patients, animals are usually free of other ocular pathologies, and their imaging under anesthesia experiences fewer motion artifacts. Compared with conventional approaches such as electroretinogram and visual field tests, optoretinography is strongly desired in clinical settings owing to its merits in non-invasiveness, objectiveness, and unprecedented spatial resolution. For example, it may serve as an ideal tool for the diagnosis/prognosis of retinal degenerative diseases at the early stage, when no clear signs of retinal degeneration are visible in structural images.

Despite these potential benefits of conducting optoretinography studies in rodents, the axial resolution of near-infrared OCT (NIR-OCT) systems, typically limited to several micrometers in tissue, hinders the clear differentiation of rodent photoreceptor OS terminals and RPE, particularly given the interdigitation of microvilli with the OS. In a typical structural image captured by NIR-OCT, for example, the OS and RPE are compounded in a speckled layer. Moreover, since the backscattered light is coherently convolved with the point spread function of the optical system, a single pixel (an individual pixel) in the OCT image can contain a mixture of phase signals from multiple types of cells. As a result, separating the optoretinography signals from different outer retinal bands (e.g., the OS and RPE) presents a significant technical challenge.

A need therefore exists to provide a method of performing optoretinography using OCT, as well as a system thereof, that seeks to overcome, or at least ameliorate, one or more deficiencies in conventional methods of performing optoretinography using OCT, and more particularly, in a manner which enables signals from different outer retinal bands to be resolved, thus enhancing/improving the accuracy (e.g., resolution) in imaging or detecting the dynamics (functional responses, such as movements or deformations) of outer retinal tissues in response to a visual stimulus, such as enabling the detection of nanometer-scale tissue dynamics. It is against this background that the present invention has been developed.

### SUMMARY

According to a first aspect of the present invention, there is provided a method of training a machine learning model for classifying a temporal phase signal obtained from optical coherence tomography (OCT) images produced for optoretinography using at least one processor, the method comprising:
obtaining a series of OCT images produced by a phase-sensitive OCT system for optoretinography with respect to an outer retina, including a stimulus sub-series of OCT images produced in response to a visual stimulus to the outer retina;
determining, for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images, a temporal phase signal of the series of corresponding individual pixels, resulting in a set of temporal phase signals for the plurality of series of corresponding individual pixels, the target layer comprising a plurality of outer retinal bands;
projecting a plurality of temporal phase signals of the set of temporal phase signals to a plurality of feature points in a feature space;
labelling each of the plurality of feature points in the feature space; and
training the machine learning model based on the plurality of labelled feature points in the feature space.

According to a second aspect of the present invention, there is provided a method of classifying a temporal phase signal obtained from OCT images produced for optoretinography using the trained machine learning model trained according to the first aspect of the present invention, the method comprising:
obtaining a series of OCT images produced by a phase-sensitive OCT system for optoretinography with respect to an outer retina, including a stimulus sub-series of OCT images produced in response to a visual stimulus to the outer retina;
determining, for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands;
projecting the temporal phase signal of the series of corresponding individual pixels to a feature point in a feature space; and
classifying the temporal phase signal using the trained machine learning model based on the feature point which the temporal phase signal has been projected to.

According to a third aspect of the present invention, there is provided a system for training a machine learning model for classifying a temporal phase signal obtained from OCT images produced for optoretinography, the system comprising:
at least one memory; and
at least one processor communicatively coupled to the at least one memory and configured to perform the method of training a machine learning model according to the first aspect of the present invention.

According to a fourth aspect of the present invention, there is provided a system for classifying a temporal phase signal obtained from OCT images produced for optoretinography, the system comprising:
at least one memory; and
at least one processor communicatively coupled to the at least one memory and configured to perform the method of classifying a temporal phase signal according to the second aspect of the present invention.

According to a fifth aspect of the present invention, there is provided a computer program product, embodied in one or more non-transitory computer-readable storage mediums, comprising instructions executable by at least one processor to perform the method of training a machine learning model according to the first aspect of the present invention.

According to a sixth aspect of the present invention, there is provided a computer program product, embodied in one or more non-transitory computer-readable storage mediums, comprising instructions executable by at least one processor to perform the method of classifying a temporal phase signal according to the second aspect of the present invention.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic flow diagram of a method of training a machine learning model for classifying a temporal phase signal obtained from OCT images produced for optoretinography, according to various embodiments of the present invention;

FIG. 2 depicts a schematic flow diagram of a method of classifying a temporal phase signal obtained from OCT images produced for optoretinography, according to various embodiments of the present invention;

FIG. 3 depicts a schematic block diagram of a system for training a machine learning model for classifying a temporal phase signal obtained from OCT images produced for optoretinography, according to various embodiments of the present invention;

FIG. 4 depicts a schematic block diagram of a system for classifying a temporal phase signal obtained from OCT images produced for optoretinography using the trained machine learning model, according to various embodiments of the present invention;

FIG. 5 depicts a schematic block diagram of an exemplary computer system which may be used to realize or implement the system for training a machine learning model and/or the system for classifying a temporal phase signal, according to various embodiments of the present invention;

FIGS. 6A to 6I illustrate an example image and signal processing pipeline/framework for the method of training a machine learning model, according to various example embodiments of the present invention;

FIG. 7A illustrates the unsupervised clustering using hierarchical clustering and subsequent classification on new OCT image dataset, according to various example embodiments of the present invention;

FIG. 7B shows the trained SVM decision boundaries for Type-I signal and Type-II signal in the PC (principal component) space, according to various example embodiments of the present invention;

FIG. 7C shows the average phase traces of the classified Type-I signals and Type-II signals, with the bands showing their standard deviations, according to various example embodiments of the present invention;

FIG. 7D shows the distribution of the peak ΔOPL and corresponding latency of individual signals in FIG. 7C, according to various example embodiments of the present invention;

FIG. 8 depicts a schematic flow diagram showing an overview of: the processing complex-valued OCT signals of OCT images to obtain temporal phase signals for training a machine learning model, the training of the machine learning model and the classification of new temporal phase signals obtained with respect to a target layer using the trained machine learning model, according to various example embodiments of the present invention;

FIGS. 9A to 9D illustrate various stages (labelled with ‘A’, ‘B’, ‘C’ and ‘D’) of the flow diagram shown in FIG. 8;

FIGS. 10A to 10D show retinal layers and their dynamics in response to visual stimuli in experiments conducted, according to various example embodiments of the present invention;

FIGS. 11A to 11D illustrate the unsupervised clustering in the spatiotemporal feature space for signal classification, according to various example embodiments of the present invention;

FIGS. 12A to 12C illustrate the classification of new phase traces and the validation of their origins, according to various example embodiments of the present invention;

FIGS. 13A to 13C illustrate the responses of the outer segment (OS) and subretinal space (SRS) in different conditions, according to various example embodiments of the present invention;

FIGS. 14A to 14C show the representative en-face functional maps of OS and SRS signals, according to various example embodiments of the present invention;

FIGS. 15A to 15C depict an example system setup and example stimulus scheme and acquisition protocols, according to various example embodiments of the present invention;

FIG. 16A shows a table (Table 1) presenting various details of animal experiments conducted, according to various example embodiments of the present invention;

FIG. 16B shows a table (Table 2) presenting various details of the acquisition and stimulation protocols, according to various example embodiments of the present invention; and

FIGS. 17A to 17E illustrate the deformation of cellular layers over time and comparison of optoretinography with ERG.

### DETAILED DESCRIPTION

Various embodiments of the present invention relate to machine learning-based optoretinography (ORG) using phase-sensitive optical coherence tomography (OCT). In this regard, in various embodiments, there is provided a method of training a machine learning model for classifying a temporal phase signal obtained from OCT images produced for optoretinography and a system thereof, as well as a method of classifying a temporal phase signal obtained from OCT images produced for optoretinography using the trained machine learning model and a system thereof.

As explained in the background, there are various drawbacks or deficiencies in conventional methods of performing optoretinography using OCT. In particular, due to limited spatial resolution, conventional methods fail to resolve the signals scattered from individual cells in various different outer retinal tissues in response to a visual stimulus, thereby exhibiting obscure speckle patterns in OCT images produced for optoretinography. Therefore, the accuracy (e.g., resolution) in imaging or detecting the dynamics (functional responses, such as movements or deformations) of outer retinal tissues in response to a visual stimulus is negatively affected by such obscure speckle patterns (which may be referred to as a speckled layer) in OCT images. In this regard, various embodiments advantageously address the technical problem associated with these speckle patterns in OCT images, and more particularly, provide a method of performing optoretinography using OCT in a manner which enables signals from various outer retinal bands to be resolved, thus enhancing/improving the accuracy (e.g., resolution) in imaging or detecting the dynamics of outer retinal tissues in response to a visual stimulus, such as enabling the detection of nanometer-scale tissue dynamics. In this regard, various embodiments provide a method of training a machine learning model for classifying a temporal phase signal obtained from OCT images produced for optoretinography and a system thereof, as well as a method of classifying a temporal phase signal obtained from OCT images produced for optoretinography using the trained machine learning model and a system thereof.

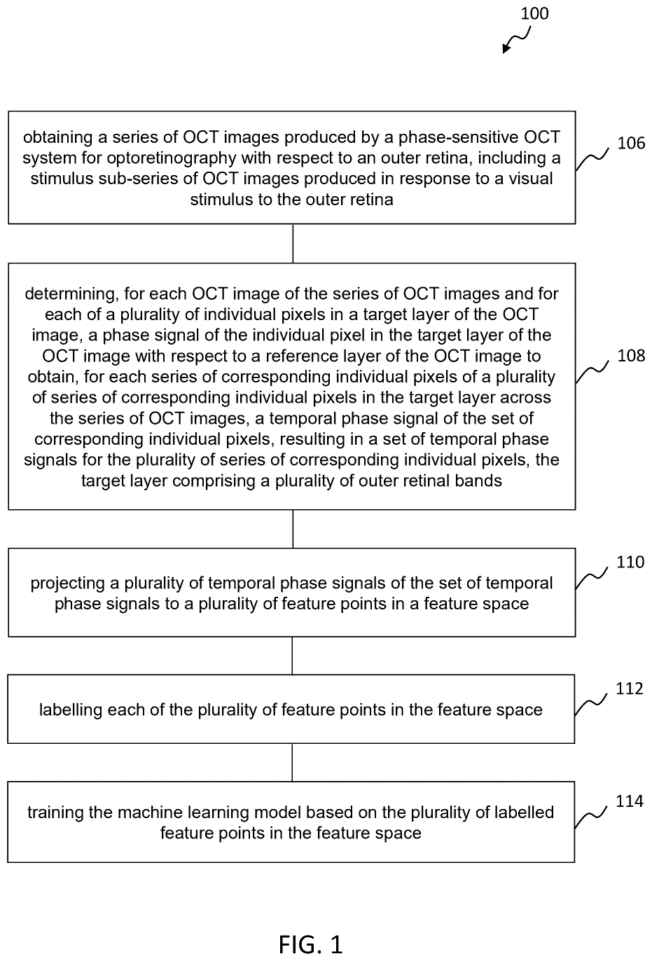

FIG. 1 depicts a schematic flow diagram of a method 100 of training a machine learning model for classifying a temporal phase signal obtained from OCT images produced for optoretinography using at least one processor, according to various embodiments of the present invention. The method 100 comprises: obtaining (at 106) a series of OCT images produced by a phase-sensitive OCT system for optoretinography with respect to an outer retina, including a stimulus sub-series of OCT images produced in response to a visual stimulus to the outer retina; determining (at 108), for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images, a temporal phase signal of the series of corresponding individual pixels, resulting in a set of temporal phase signals for the plurality of series of corresponding individual pixels, the target layer comprising a plurality of outer retinal bands; projecting (at 110) a plurality of temporal phase signals of the set of temporal phase signals to a plurality of feature points in a feature space; labelling (at 112) each of the plurality of feature points in the feature space; and training (at 114) the machine learning model based on the plurality of labelled feature points in the feature space.

Accordingly, the method 100 of training a machine learning model for classifying a temporal phase signal obtained from OCT images is advantageously able to resolve signals from various outer retinal bands, thus enhancing/improving the accuracy (e.g., resolution) in imaging or detecting the dynamics of outer retinal tissues in response to a visual stimulus. In particular, for each series of corresponding individual pixels (one corresponding individual pixel from each OCT image across the series of OCT images) of a plurality of series of corresponding individual pixels in the target layer across the series of OCT images, a temporal phase signal of the series of corresponding individual pixels is determined, resulting in a set of temporal phase signals for the plurality of series of corresponding individual pixels (e.g., with respect to each individual pixel in the target layer). A plurality of temporal phase signals of the set of temporal phase signals are then projected to a plurality of feature points in a feature space (e.g., a temporal feature space or a spatiotemporal feature space) such that the plurality of feature points may be grouped into a plurality of clusters (each cluster having a respective label assigned thereto, which may correspond to one of a plurality of outer retinal tissue types or an outlier type) and a machine learning model may then be trained based on the plurality of labelled feature points in the feature space for classifying a new temporal phase signal. Such an approach has been advantageously found to address the above-described technical problem associated with speckle patterns (e.g., a speckled layer, e.g., corresponding to the above-mentioned target layer) in OCT images which negatively affected the accuracy (e.g., resolution) in imaging or detecting the dynamics of outer retinal tissues in response to a visual stimulus. Therefore, training a machine learning model for classifying a temporal phase signal according to the method 100 advantageously allows machine learning-based optoretinography using phase-sensitive OCT to be performed in a manner which enables signals (temporal phase signals) from various different outer retinal bands in the speckled layer (e.g., corresponding to the above-mentioned target layer) to be resolved, thus enhancing/improving the accuracy (e.g., resolution) in imaging or detecting the dynamics of outer retinal tissues in response to a visual stimulus, such as enabling the detection of nanometer-scale tissue dynamics. These advantages or technical effects, and/or other advantages or technical effects, will become more apparent to a person skilled in the art as the method 100 of training a machine learning model and the method of classifying a temporal phase signal using the trained machine learning model (to be described later below), as well as the corresponding systems, are described in more detail according to various embodiments and example embodiments of the present invention.

In various embodiments, the above-mentioned determining (at 108) the phase signal of the individual pixel in the target layer of the OCT image comprises correcting a systematic phase drift of a complex-valued OCT signal of the individual pixel based on self-referencing to an individual pixel of the reference layer of the OCT image to obtain a first complex-valued OCT signal of the individual pixel.

In various embodiments, the above-mentioned correcting the systematic phase drift of the complex-valued OCT signal of the individual pixel comprises multiplying the complex-valued OCT signal of the individual pixel with a complex conjugate of the complex-valued OCT signal of the individual pixel of the reference layer of the OCT image to obtain the first complex-valued OCT signal of the individual pixel.

In various embodiments, for each OCT image of the stimulus sub-series of OCT images, the above-mentioned determining (at 108) the phase signal of the individual pixel in the target layer of the OCT image further comprises correcting a phase offset of the first complex-valued OCT signal of the individual pixel based on a pre-stimulus sub-series of the series of OCT images produced prior to the visual stimulus to the outer retina to obtain a second complex-valued OCT signal of the individual pixel.

In various embodiments, the above-mentioned correcting the phase offset of the first complex-valued OCT signal of the individual pixel comprises: determining an average of the first complex-valued OCT signals of corresponding individual pixels of the pre-stimulus sub-series of OCT images corresponding to the individual pixel of the OCT image to obtain an averaged pre-stimulus complex-valued OCT signal; and multiplying the first complex-valued OCT signal of the individual pixel with the averaged pre-stimulus complex-valued OCT signal to obtain the second complex-valued OCT signal of the individual pixel.

In various embodiments, for each OCT image of the stimulus sub-series of OCT images, the phase signal of the individual pixel in the target layer of the OCT image is determined based on the second complex-valued OCT signal of the individual pixel. In various embodiments, similarly, for each OCT image of the pre-stimulus sub-series of OCT images, the above-mentioned determining (at 108) the phase signal of the individual pixel in the target layer of the OCT image may also comprises correcting a phase offset of the first complex-valued OCT signal of the individual pixel based on the pre-stimulus sub-series of the series of OCT images to obtain a second complex-valued OCT signal of the individual pixel and determining the phase signal of the individual pixel in the target layer of the OCT image in the same or similar manner as described above for each OCT image of the stimulus sub-series of OCT images.

In various embodiments, the phase signal of the individual pixel in the target layer of the OCT image is determined with respect to a reference pixel region of the reference layer of the OCT image, the reference pixel region comprising a plurality of individual pixels. In this regard, for the above-mentioned correcting the systematic phase drift of the complex-valued OCT signal of the individual pixel, the above-mentioned multiplying the complex-valued OCT signal of the individual pixel comprises multiplying the complex-valued OCT signal of the individual pixel respectively with complex conjugates of the complex-valued OCT signals of the plurality of individual pixels of the reference pixel region of the reference layer of the OCT image to obtain a set of first complex-valued OCT signals of the individual pixel. In addition, for the above-mentioned correcting the phase offset of the complex-valued OCT signal of the individual pixel, the above-mentioned determining the average of the first complex-value OCT signals of corresponding individual pixels comprises determining an average of the sets of first complex-valued OCT signals of corresponding individual pixels of the pre-stimulus sub-series of OCT images corresponding to the individual pixel of the OCT image to obtain an averaged set of pre-stimulus complex-valued OCT signals; and the above-mentioned multiplying the first complex-valued OCT signal of the individual pixel comprises multiplying the set of first complex-valued OCT signals of the individual pixel with the averaged set of pre-stimulus complex-valued OCT signals to obtain a set of second complex-valued OCT signals of the individual pixel.

In various embodiments, in the case that the phase signal of the individual pixel in the target layer of the OCT image is determined with respect to the reference pixel region of the reference layer of the OCT image, for each OCT image of the stimulus sub-series of OCT images, the above-mentioned determining (at 108) the phase signal of the individual pixel in the target layer of the OCT image further comprises: determining an average of the set of second complex-valued OCT signals of the individual pixel to obtain an averaged complex-valued OCT signal of the individual pixel; and determining the phase signal of the individual pixel in the target layer of the OCT image based on the averaged complex-valued OCT signal of the individual pixel. In various embodiments, similarly, for each OCT image of the pre-stimulus sub-series of OCT images, the above-mentioned determining (at 108) the phase signal of the individual pixel in the target layer of the OCT image further comprises: determining an average of the set of second complex-valued OCT signals of the individual pixel to obtain an averaged complex-valued OCT signal of the individual pixel; and determining the phase signal of the individual pixel in the target layer of the OCT image based on the averaged complex-valued OCT signal of the individual pixel.

In various embodiments, the temporal phase signal of the series of corresponding individual pixels in the target layer across the series of OCT images is obtained based on the phase signals of the series of corresponding individual pixels in the target layer across the series of OCT images.

In various embodiments, the above-mentioned labelling (at 112) each of the plurality of feature points in the feature space comprises: grouping the plurality of feature points into a plurality of clusters in the feature space; and labelling, for each of the plurality of clusters, feature points belonging to the cluster with a label assigned to the cluster.

In various embodiments, the plurality of feature points is grouped into the plurality of clusters based on an unsupervised clustering technique.

In various embodiments, the plurality of clusters has a plurality of different labels assigned thereto, respectively, the plurality of different labels comprising a plurality of different outer retinal band labels corresponding to the plurality of outer retinal bands of the target layer.

In various embodiments, the reference layer corresponds to the inner segment/outer segment (IS/OS) junction, and the plurality of outer retinal bands comprises two or more outer retinal bands respectively corresponding to two or more of a photoreceptor outer segment, a microvilli, a retinal pigment epithelium (RPE) and a Bruch's membrane (BrM).

In various embodiments, the method 100 further comprises, prior to projecting the plurality of temporal phase signals into the plurality of feature points in the feature space, subjecting the plurality of temporal phase signals to a bandstop filter and a low-pass filter; and normalizing the plurality of temporal phase signals.

In various embodiments, the feature space is a temporal feature space or a spatiotemporal feature space.

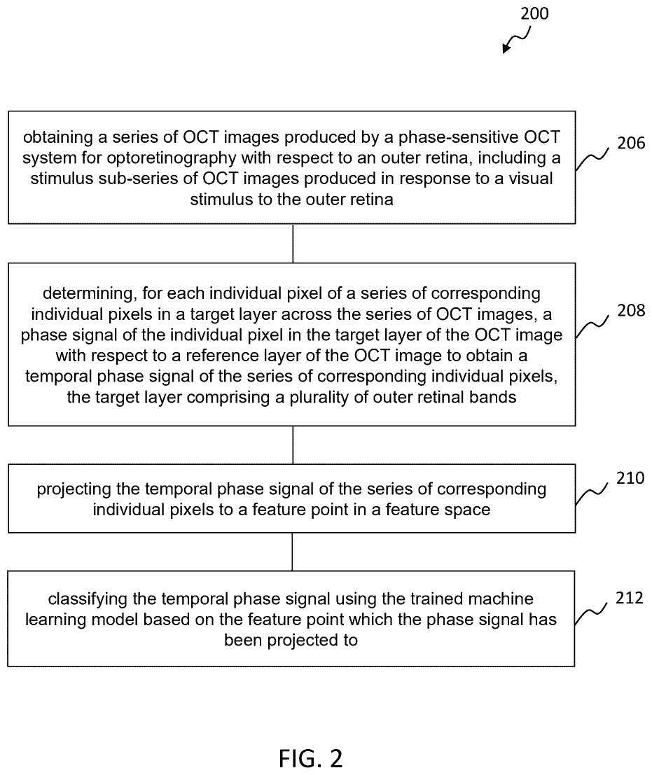

FIG. 2 depicts a schematic flow diagram of a method 200 of classifying a temporal phase signal obtained from OCT images produced for optoretinography, according to various embodiments of the present invention, using the trained machine learning model trained according to the method 100 according to various embodiments of the present invention. The method 200 comprises: obtaining (at 206) a series of OCT images produced by a phase-sensitive OCT system for optoretinography with respect to an outer retina, including a stimulus sub-series of OCT images produced in response to a visual stimulus to the outer retina; determining (at 208), for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands; projecting (at 210) the temporal phase signal of the series of corresponding individual pixels to a feature point in a feature space; and classifying (at 212) the temporal phase signal using the trained machine learning model based on the feature point which the temporal phase signal has been projected to.

Accordingly, the above-mentioned obtaining (at 206) the series of OCT images, the above-mentioned determining (at 208) the phase signal of the individual pixel in the target layer of the OCT image and the above-mentioned projecting (at 210) the temporal phase signal of the series of corresponding individual pixels to a feature point in the feature space for the method 200 of classifying a temporal phase signal may be performed in the same or similar manner as the above-mentioned obtaining (at 106) the series of OCT images, the above-mentioned determining (at 108) the phase signal of the individual pixel in the target layer of the OCT image and the above-mentioned projecting (at 110) the plurality of temporal phase signals of the series of corresponding individual pixels to a plurality of feature points in the feature space for the method 100 of training a machine learning model as described hereinbefore according to various embodiments of the present invention, respectively. Furthermore, in various embodiments, the above-mentioned projecting (at 210) the temporal phase signal of the series of corresponding individual pixels to a feature point in the feature space is the same feature space as utilized for training the machine learning model. In various embodiments, the feature space is a temporal feature space or a spatiotemporal feature space.

In various embodiments, the above-mentioned determining (at 208) the phase signal of the individual pixel in the target layer of the OCT image comprises correcting a systematic phase drift of a complex-valued OCT signal of the individual pixel based on self-referencing to an individual pixel of the reference layer of the OCT image to obtain a first complex-valued OCT signal of the individual pixel.

In various embodiments, the above-mentioned correcting the systematic phase drift of the complex-valued OCT signal of the individual pixel comprises multiplying the complex-valued OCT signal of the individual pixel with a complex conjugate of the complex-valued OCT signal of the individual pixel of the reference layer of the OCT image to obtain the first complex-valued OCT signal of the individual pixel.

In various embodiments, for each OCT image of the stimulus sub-series of OCT images, the above-mentioned determining (at 208) the phase signal of the individual pixel in the target layer of the OCT image further comprises correcting a phase offset of the first complex-valued OCT signal of the individual pixel based on a pre-stimulus sub-series of the series of OCT images produced prior to the visual stimulus to the outer retina to obtain a second complex-valued OCT signal of the individual pixel.

In various embodiments, the above-mentioned correcting the phase offset of the first complex-valued OCT signal of the individual pixel comprises: determining an average of the first complex-valued OCT signals of corresponding individual pixels of the pre-stimulus sub-series of OCT images corresponding to the individual pixel of the OCT image to obtain an averaged pre-stimulus complex-valued OCT signal; and multiplying the first complex-valued OCT signal of the individual pixel with the averaged pre-stimulus complex-valued OCT signal to obtain the second complex-valued OCT signal of the individual pixel.

In various embodiments, for each OCT image of the stimulus sub-series of OCT images, the phase signal of the individual pixel in the target layer of the OCT image is determined based on the second complex-valued OCT signal of the individual pixel. In various embodiments, similarly, for each OCT image of the pre-stimulus sub-series of OCT images, the above-mentioned determining (at 208) the phase signal of the individual pixel in the target layer of the OCT image may also comprises correcting a phase offset of the first complex-valued OCT signal of the individual pixel based on the pre-stimulus sub-series of the series of OCT images to obtain a second complex-valued OCT signal of the individual pixel and determining the phase signal of the individual pixel in the target layer of the OCT image in the same or similar manner as described above for each OCT image of the stimulus sub-series of OCT images.

In various embodiments, the phase signal of the individual pixel in the target layer of the OCT image is determined with respect to a reference pixel region of the reference layer of the OCT image, the reference pixel region comprising a plurality of individual pixels. In this regard, for the above-mentioned correcting the systematic phase drift of the complex-valued OCT signal of the individual pixel, the above-mentioned multiplying the complex-valued OCT signal of the individual pixel comprises multiplying the complex-valued OCT signal of the individual pixel respectively with complex conjugates of the complex-valued OCT signals of the plurality of individual pixels of the reference pixel region of the reference layer of the OCT image to obtain a set of first complex-valued OCT signals of the individual pixel. In addition, for the above-mentioned correcting the phase offset of the complex-valued OCT signal of the individual pixel, the above-mentioned determining the average of the first complex-value OCT signals of corresponding individual pixels comprises determining an average of the sets of first complex-valued OCT signals of corresponding individual pixels of the pre-stimulus sub-series of OCT images corresponding to the individual pixel of the OCT image to obtain an averaged set of pre-stimulus complex-valued OCT signals, and the above-mentioned multiplying the first complex-valued OCT signal of the individual pixel comprises multiplying the set of first complex-valued OCT signals of the individual pixel with the averaged set of pre-stimulus complex-valued OCT signals to obtain a set of second complex-valued OCT signals of the individual pixel.

In various embodiments, in the case that the phase signal of the individual pixel in the target layer of the OCT image is determined with respect to the reference pixel region of the reference layer of the OCT image, for each OCT image of the stimulus sub-series of OCT images, the above-mentioned determining (208) the phase signal of the individual pixel in the target layer of the OCT image further comprises: determining an average of the set of second complex-valued OCT signals of the individual pixel to obtain an averaged complex-valued OCT signal of the individual pixel; and determining the phase signal of the individual pixel in the target layer of the OCT image based on the averaged complex-valued OCT signal of the individual pixel. In various embodiments, similarly, for each OCT image of the pre-stimulus sub-series of OCT images, the above-mentioned determining (at 208) the phase signal of the individual pixel in the target layer of the OCT image further comprises: determining an average of the set of second complex-valued OCT signals of the individual pixel to obtain an averaged complex-valued OCT signal of the individual pixel; and determining the phase signal of the individual pixel in the target layer of the OCT image based on the averaged complex-valued OCT signal of the individual pixel.

In various embodiments, the temporal phase signal of the series of corresponding individual pixels in the target layer across the series of OCT images is obtained based on the phase signals of the series of corresponding individual pixels in the target layer across the series of OCT images.

In various embodiments, the temporal phase signal is classified using the trained machine learning model into one of a plurality of outer retinal tissue types corresponding to a plurality of outer retinal bands of the target layer or an outlier type.

In various embodiments, the reference layer corresponds to the inner segment/outer segment (IS/OS) junction, and the plurality of outer retinal bands comprises two or more outer retinal bands respectively corresponding to two or more of a photoreceptor outer segment, a microvilli, a retinal pigment epithelium (RPE) and a Bruch's membrane (BrM).

In various embodiments, the method 200 further comprises, prior to projecting the temporal phase signal to the feature point in the feature space, subjecting the temporal phase signal to a bandstop filter and a low-pass filter; and normalizing the temporal phase signal.

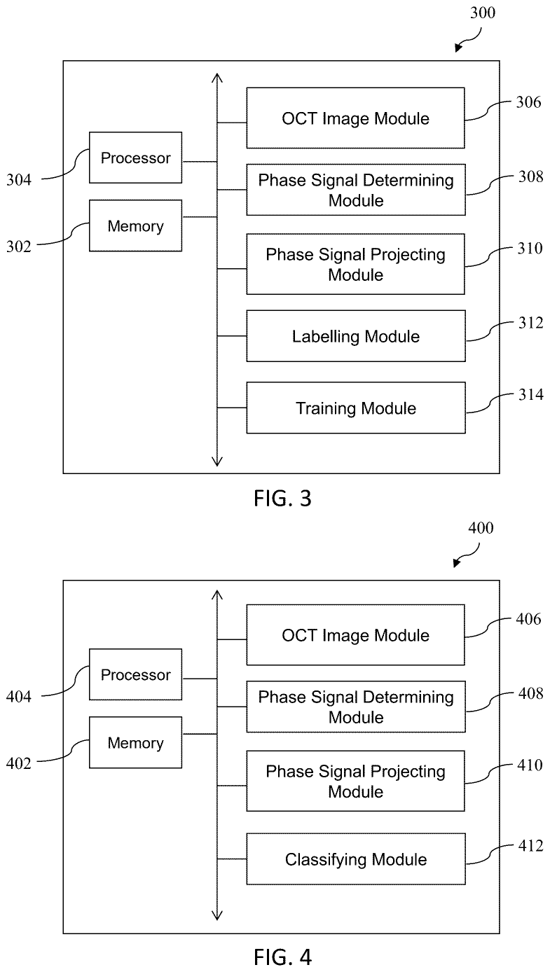

FIG. 3 depicts a schematic block diagram of a system 300 for training a machine learning model for classifying a temporal phase signal obtained from OCT images produced for optoretinography, according to various embodiments of the present invention, corresponding to the above-mentioned method 100 of training a machine learning model as described hereinbefore according with reference to FIG. 1 according to various embodiments of the present invention. The system 300 comprising: at least one memory 302; and at least one processor 304 communicatively coupled to the at least one memory 302 and configured to perform the method 100 of training a machine learning model as described hereinbefore according to various embodiments of the present invention. Accordingly, the at least one processor 304 is configured to: obtain a series of OCT images produced by a phase-sensitive OCT system for optoretinography with respect to an outer retina, including a stimulus sub-series of OCT images produced in response to a visual stimulus to the outer retina; determine, for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images, a temporal phase signal of the series of corresponding individual pixels, resulting in a set of temporal phase signals for the plurality of series of corresponding individual pixels, the target layer comprising a plurality of outer retinal bands; project a plurality of temporal phase signals of the set of temporal phase signals into a plurality of feature points in a feature space; label each of the plurality of temporal phase signals in the feature space; and train the machine learning model based on the plurality of labelled temporal phase signals in the feature space.

It will be appreciated by a person skilled in the art that the at least one processor 304 may be configured to perform various functions or operations through set(s) of instructions (e.g., software modules) executable by the at least one processor 304 to perform various functions or operations. Accordingly, as shown in FIG. 3, the system 300 may comprise: an OCT image module (or an OCT image circuit) 306 configured to obtain a series of OCT images produced by a phase-sensitive OCT system for optoretinography with respect to an outer retina, including a stimulus sub-series of OCT images produced in response to a visual stimulus to the outer retina; a phase signal determining (or extracting) module (or a phase signal determining (extracting) circuit) 308 configured to determine, for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images, a temporal phase signal of the series of corresponding individual pixels, resulting in a set of temporal phase signals for the plurality of series of corresponding individual pixels, the target layer comprising a plurality of outer retinal bands; a phase signal projecting module (or a phase signal projecting circuit) 310 configured to project a plurality of temporal phase signals of the set of temporal phase signals to a plurality of feature points in a feature space; a labelling module (or a labelling circuit) 312 configured to label each of the plurality of feature points in the feature space; and a training module (or a training circuit) 314 configured to train the machine learning model based on the plurality of labelled feature points in the feature space.

It will be appreciated by a person skilled in the art that the above-mentioned modules of the system 300 are not necessarily separate modules, and two or more modules may be realized by or implemented as one functional module (e.g., a circuit or a software program) as desired or as appropriate without deviating from the scope of the present invention. For example, two or more of the OCT image module 306; the phase signal determining module 308; the phase signal projecting module 310; the labelling module 312 and the training module 314 may be realized (e.g., compiled together) as one executable software program (e.g., software application or simply referred to as an “app”), which for example may be stored in the at least one memory 302 and executable by the at least one processor 304 to perform the corresponding functions or operations as described herein according to various embodiments.

In various embodiments, the system 300 for training a machine learning model corresponds to the method 100 of training a machine learning model as described hereinbefore with reference to FIG. 1, therefore, various operations, functions or steps configured to be performed by the least one processor 304 may correspond to various operations, functions or steps of the method 100 described hereinbefore according to various embodiments, and thus need not be repeated with respect to the system 300 for clarity and conciseness. In other words, various embodiments described herein in context of methods (e.g., the method 100) are analogously valid for the corresponding systems or devices (e.g., the system 300), and vice versa. For example, in various embodiments, the at least one memory 302 may have stored therein the OCT image module 306; the phase signal determining module 308; the phase signal projecting module 310; the labelling module 312 and/or the training module 314, which respectively correspond to various operations, functions or steps of the method 100 as described hereinbefore according to various embodiments, which are executable by the at least one processor 304 to perform the corresponding operations, functions or steps as described herein.

FIG. 4 depicts a schematic block diagram of a system 400 for classifying a temporal phase signal obtained from OCT images produced for optoretinography using the trained machine learning model trained according to the method 100 or by the system 300 according to various embodiments of the present invention, corresponding to the above-mentioned method 200 of classifying a temporal phase signal as described hereinbefore according with reference to FIG. 2 according to various embodiments of the present invention. The system 400 comprising: at least one memory 402; and at least one processor 404 communicatively coupled to the at least one memory 402 and configured to perform the method 200 of classifying a temporal phase signal as described hereinbefore according to various embodiments of the present invention. Accordingly, the at least one processor 404 is configured to: obtain a series of OCT images produced by a phase-sensitive OCT system for optoretinography with respect to an outer retina, including a stimulus sub-series of OCT images produced in response to a visual stimulus to the outer retina; determine, for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands; project the temporal phase signal of the series of corresponding individual pixels to a feature point in a feature space; and classify the temporal phase signal using the trained machine learning model based on the feature point which the temporal phase signal has been projected to.

Similarly, it will be appreciated by a person skilled in the art that the at least one processor 404 may be configured to perform various functions or operations through set(s) of instructions (e.g., software modules) executable by the at least one processor 404 to perform various functions or operations. Accordingly, as shown in FIG. 4, the system 400 may comprise: an OCT image module (or an OCT image circuit) 406 configured to obtain a series of OCT images produced by a phase-sensitive OCT system for optoretinography with respect to an outer retina, including a stimulus sub-series of OCT images produced in response to a visual stimulus to the outer retina; a phase signal determining (or extracting) module (or a phase signal determining (extracting) circuit) 408 configured to determine, for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands; a phase signal projecting module (or a phase signal projecting circuit) 410 configured to project the temporal phase signal of the series of corresponding individual pixels to a feature point in a feature space; and a classifying module (or a classifying circuit) 412 configured to classify the temporal phase signal using the trained machine learning model based on the feature point which the temporal phase signal has been projected to.

Similarly, it will be appreciated by a person skilled in the art that the above-mentioned modules of the system 400 are not necessarily separate modules, and two or more modules may be realized by or implemented as one functional module (e.g., a circuit or a software program) as desired or as appropriate without deviating from the scope of the present invention. For example, two or more of the OCT image module 406; the phase signal determining module 408; the phase signal projecting module 410 and the classifying module 412 may be realized (e.g., compiled together) as one executable software program (e.g., software application or simply referred to as an “app”), which for example may be stored in the at least one memory 402 and executable by the at least one processor 404 to perform the corresponding functions or operations as described herein according to various embodiments.

In various embodiments, the system 400 for classifying a temporal phase signal corresponds to the method 400 of classifying a temporal phase signal as described hereinbefore with reference to FIG. 2, therefore, various operations, functions or steps configured to be performed by the least one processor 404 may correspond to various operations, functions or steps of the method 200 described hereinbefore according to various embodiments, and thus need not be repeated with respect to the system 400 for clarity and conciseness. In other words, various embodiments described herein in context of methods (e.g., the method 200) are analogously valid for the corresponding systems or devices (e.g., the system 400), and vice versa. For example, in various embodiments, the at least one memory 402 may have stored therein the OCT image module 406; the phase signal determining module 408; the phase signal projecting module 410 and/or the classifying module 412, which respectively correspond to various operations, functions or steps of the method 200 as described hereinbefore according to various embodiments, which are executable by the at least one processor 404 to perform the corresponding operations, functions or steps as described herein.

In various embodiments, the system 300 and the system 400 may be implemented or integrated as one system (e.g., the OCT image module 406, the phase signal determining module 408 and the phase signal projecting module 410 may be the same as the OCT image module 306, the phase signal determining module 308 and the phase signal projecting module 310, respectively). Accordingly, the integrated/combined system may comprise the OCT image module 306/406, the phase signal determining module 308/408, the phase signal projecting module 310/410, the labelling module 312, the training module 314 and the classifying module 412.

A computing system, a controller, a microcontroller or any other system providing a processing capability may be provided according to various embodiments in the present invention. Such a system may be taken to include one or more processors and one or more computer-readable storage mediums. For example, the system 300 and the system 400 described hereinbefore may include at least one processor (or controller) and at least one computer-readable storage medium (or memory) which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of various functions or operations may also be understood as a “circuit” in accordance with various other embodiments. Similarly, a “module” may be a portion of a system according to various embodiments in the present invention and may encompass a “circuit” as above, or may be understood to be any kind of a logic-implementing entity therefrom.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

The present specification also discloses a system (e.g., which may also be embodied as one or more devices or apparatuses), such as the system 300 and the system 400, for performing various operations, functions or steps of various methods described herein. Such a system may be specially constructed for the required purposes or may comprise a general purpose computer system selectively activated or reconfigured by a computer program stored in the computer system. In general, various algorithms that may be presented herein are not limited to being implemented or executed by any particular computer system. Alternatively, the construction of more specialized computer system to perform various operations, functions or steps of various methods described herein may be provided as desired or as appropriate without going beyond the scope of the present invention.

In addition, the present specification also at least implicitly discloses computer program(s) or software/functional module(s), in that it would be apparent to a person skilled in the art that various operations, functions or steps of various methods described herein may be put into effect by computer code. The computer program(s) is not intended to be limited to any particular programming language and implementation thereof, and it will be appreciated by a person skilled in the art that a variety of programming languages and coding thereof may be used to implement the computer program(s). Moreover, the computer program(s) is not intended to be limited to any particular control flow as there are a variety of programming languages which can use different control flows. It will be appreciated by a person skilled in the art that a computer program may be stored on any computer-readable storage medium (non-transitory computer-readable storage medium), such as but not limited to, a magnetic disk, an optical disk or a memory chip. For example, a computer program stored on a computer-readable storage medium may be loaded and executed on a computer system to implement various operations, functions or steps of various methods described herein according to various embodiments of the present invention.

Accordingly, in various embodiments, there is provided a computer program product, embodied in one or more computer-readable storage mediums (non-transitory computer-readable storage medium), comprising instructions (e.g., the OCT image module 306; the phase signal determining module 308; the phase signal projecting module 310; the labelling module 312 and/or the training module 314) executable by one or more computer processors to perform the method 100 of training a machine learning model as described hereinbefore with reference to FIG. 1 according to various embodiments of the present invention. Accordingly, various computer programs or software modules described herein may be stored in a computer program product receivable by a system therein, such as the system 300 as shown in FIG. 3, for execution by at least one processor 304 of the system 300 to perform various operations, functions or steps of various methods described herein according to various embodiments of the present invention. Similarly, in various embodiments, there is provided a computer program product, embodied in one or more computer-readable storage mediums (non-transitory computer-readable storage medium), comprising instructions (e.g., the OCT image module 406; the phase signal determining module 408; the phase signal projecting module 410 and/or the classifying module 412) executable by one or more computer processors to perform the method 200 of classifying the temporal phase signal as described hereinbefore with reference to FIG. 2 according to various embodiments of the present invention. Accordingly, various computer programs or software modules described herein may be stored in a computer program product receivable by a system therein, such as the system 400 as shown in FIG. 4 for execution by at least one processor 404 of the system 400 to perform various operations, functions or steps of various methods described herein according to various embodiments of the present invention. Still further in various embodiments, there may be provided a computer program product, embodied in one or more computer-readable storage mediums (non-transitory computer-readable storage medium), comprising instructions (e.g., the OCT image module 306/406; the phase signal determining module 308/408; the phase signal projecting module 310/410; the labelling module 312, the training module 314 and classifying module 412) executable by one or more computer processors to perform the method 100 of training a machine learning model and to perform the method 200 of classifying the temporal phase signal as described hereinbefore according to various embodiments of the present invention.

It will be appreciated by a person skilled in the art that various modules of the system 300 described herein (e.g., the OCT image module 306; the phase signal determining module 308; the phase signal projecting module 310; the labelling module 312 and/or the training module 314) may be software module(s) realized by computer program(s) or set(s) of instructions executable by a computer processor to perform various functions or operations. Various modules described herein (e.g., the OCT image module 306; the phase signal determining module 308; the phase signal projecting module 310; the labelling module 312 and/or the training module 314) may also be implemented as hardware module(s) being functional hardware unit(s) designed to perform various functions or operations. Similarly, it will be appreciated by a person skilled in the art that various modules of the system 400 described herein (e.g., the OCT image module 406; the phase signal determining module 408; the phase signal projecting module 410 and/or the classifying module 412) may be software module(s) realized by computer program(s) or set(s) of instructions executable by a computer processor to perform various functions or operations. Various modules described herein (e.g., the OCT image module 406; the phase signal determining module 408; the phase signal projecting module 410 and/or the classifying module 412) may also be implemented as hardware module(s) being functional hardware unit(s) designed to perform various functions or operations. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. It will also be appreciated by a person skilled in the art that a combination of hardware and software modules may be implemented. Furthermore, various operations, functions or steps of various methods described herein may be performed in parallel rather than sequentially as desired or as appropriate (e.g., as long as it does not render the method(s) inoperable or unsatisfactory for its intended purpose).

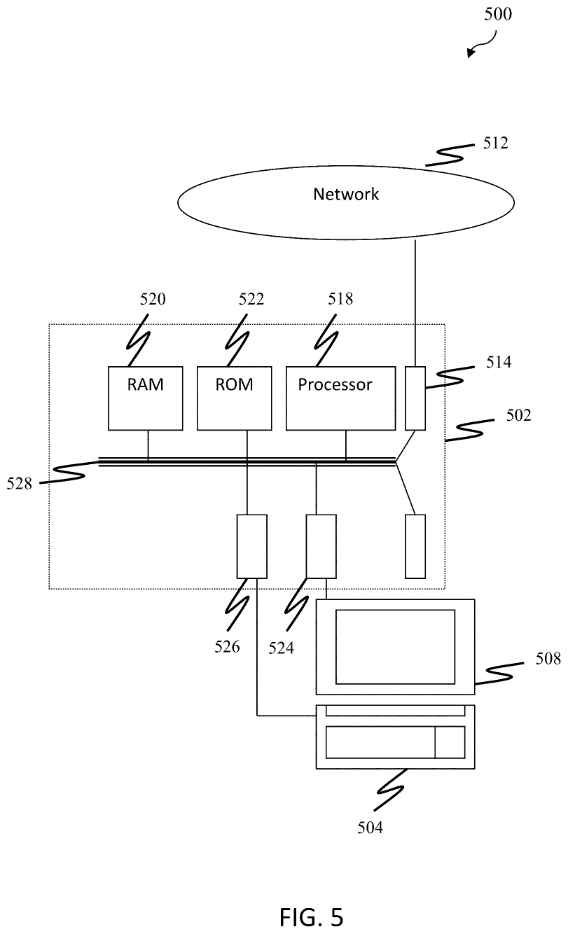

In various embodiments, the system 300 and the system 400 may be realized by any computer system (e.g., desktop or portable computer system) including at least one processor and at least one memory, such as an example computer system 500 as schematically shown in FIG. 5 as an example only and without limitation. In various embodiments, as described hereinbefore, the system 300 and the system 400 may be realized or integrated as one computer system. Various methods/steps or functional modules may be implemented as software, such as a computer program being executed within the computer system 500, and instructing the computer system 500 (in particular, one or more processors therein) to conduct various functions or operations as described herein according to various embodiments. For example, the computer system 500 may comprise a system unit 502, one or more input devices 504 such as a keyboard, a touchscreen and/or a mouse, and a plurality of output devices such as a display 508. The system unit 502 may be connected to a computer network 512 via a suitable transceiver device 514, to enable access to e.g., the Internet or other network systems such as Local Area Network (LAN) or Wide Area Network (WAN). The system unit 502 may include a processor 518 for executing various instructions, a Random Access Memory (RAM) 520 and a Read Only Memory (ROM) 522. The system unit 502 may further include a number of Input/Output (I/O) interfaces, for example I/O interface 524 to the display device 508 and I/O interface 526 to the one or more input devices 504. The components of the system unit 502 typically communicate via an interconnected bus 528 and in a manner known to a person skilled in the art.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Any reference to an element or a feature herein using a designation such as “first”, “second” and so forth does not limit the quantity or order of such elements or features, unless stated or the context requires otherwise. For example, such designations may be used herein as a convenient way of distinguishing between two or more elements or instances of an element. Thus, a reference to first and second elements does not necessarily mean that only two elements can be employed, or that the first element must precede the second element, unless stated or the context requires otherwise. In addition, a phrase referring to “at least one of” a list of items refers to any single item therein or any combination of two or more items therein.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

Various example embodiments provide a method for automated, unbiased optoretinography with spatial heterogeneity that is applicable to preclinical ocular imaging studies. In this regard, according to various example embodiments, light-evoked deformations in rod photoreceptors, pigment epithelium and subretinal space are revealed by prolonged and multilayered optoretinography (in particular, imaging functional activity of multiple outer retinal bands). As an illustrative example, wide-field, automated, unbiased optoretinography according to various example embodiments reveals comprehensive nanoscopic dynamics (nanometer-scale tissue dynamics) of the outer retina in rodents.

As explained in the background, there are various drawbacks or deficiencies in conventional methods of performing optoretinography using OCT. In particular, due to limited spatial resolution, conventional methods fail to resolve the signals scattered from individual cells in various different outer retinal tissues in response to a visual stimulus, thereby exhibiting obscure speckle patterns in OCT images produced for optoretinography. Therefore, the accuracy (e.g., resolution) in imaging or detecting the dynamics (functional responses, such as movements or deformations) of outer retinal tissues in response to a visual stimulus is negatively affected by such obscure speckle patterns (which may be referred to as a speckled layer) in OCT images. In this regard, various example embodiments advantageously address the technical problem associated with these speckle patterns in OCT images, and more particularly, provide a method of performing optoretinography using OCT in a manner which enables signals from different outer retinal bands to be resolved, thus enhancing/improving the accuracy (e.g., resolution) in imaging or detecting the dynamics of outer retinal tissues in response to a visual stimulus, such as enabling the detection of nanometer-scale tissue dynamics. In this regard, various example embodiments provide a method of training a machine learning model for classifying a temporal phase signal obtained from OCT images produced for optoretinography and a system thereof, as well as a method of classifying a temporal phase signal obtained from OCT images produced for optoretinography using the trained machine learning model and a system thereof.

Phototransduction involves changes in concentration of ions and other solutes within photoreceptors and in subretinal space, which affect osmotic pressure and the associated water flow. Corresponding expansion and contraction of cellular layers can be imaged using optoretinography, based on phase-sensitive OCT. Until now, optoretinography could reliably detect only photoisomerization and phototransduction in photoreceptors, primarily in cones under very bright stimuli. By employing a subpixel bulk motion correction method or algorithm, which enabled imaging of the nanometer-scale tissue dynamics during minute-long recordings, and unsupervised learning of spatiotemporal patterns, various example embodiments discovered optical signatures of the other retinal structures' response to visual stimuli. These outer retinal structures include inner and outer segments of rod photoreceptors, retinal pigment epithelium (RPE), and subretinal space in general. The high sensitivity of the method of performing optoretinography according to various example embodiments of the present invention enables detection of the retinal responses to very dim stimuli, e.g., down to 0.01% bleach level, corresponding to natural levels of scotopic illumination. Various example embodiments also demonstrated that with a single flash, the optoretinogram can map retinal responses across, for example, a 12° field of view, potentially replacing multifocal electroretinography, with its long acquisition time and low spatial resolution. Accordingly, the method of performing optoretinography according to various example embodiments of the present invention expands the diagnostic capabilities and practical applicability of optoretinography, providing a more complete replacement of electroretinography, while combining structural and functional retinal imaging in the same OCT machine or system. In particular, various example embodiments provide a robust, unsupervised learning approach to finding the hidden spatiotemporal patterns in the phase signals measured from speckles in OCT images, which reveals distinct signatures of, for example, the subretinal space (SRS), photoreceptor inner segment (IS) and outer segment (OS), and RPE responses to light (a visual stimulus). For example, image registration using the phase-restoring subpixel motion correction method or algorithm according to various example embodiments enables optoretinography recordings down to nanometer-scale for tens of seconds, thereby allowing for the detection of much slower and more subtle phenomena in retinal responses to a visual stimulus. Various example embodiments demonstrate these light-evoked responses under various scotopic and photopic conditions and map the OS and SRS dynamics across a wide field using a single flash.

For example, small animals, such as rodents, serve as an attractive alternative for investigating the fingerprints of photoreceptor degeneration due to their wide availability and versatility in disease models and gene manipulations. Such an advantage of small animal models may also apply to optoretinography, an emerging optical imaging modality that can measure the photoreceptor function in vivo. However, due to the extreme difficulty in resolving individual photoreceptors in small animals like rodents, there was a lack of an objective and quantitative approach that could measure their outer retina dynamics while keeping the spatial heterogeneity. Such a challenge precludes further investigation of the rod optoretinography signal and the studying of its correlation with early-stage photoreceptor degeneration. In this regard, as an illustrative example, various example embodiments demonstrate an automated, unbiased optoretinography for rodent imaging based on unsupervised machine learning. The approach according to various example embodiments automatically searches for and classifies the optoretinography (ORG) signals (or more particularly, temporal phase signal, which may also be referred to as a phase trace signal or simply as a phase trace) from the obscure speckle patterns of the outer retina captured by a low-cost, phase-sensitive OCT. Using this approach, various example embodiments observed reproducible, comprehensive nanoscopic dynamics of the outer retina in response to visual stimuli in rats. Various example embodiments further categorized or classified these optoretinography signals into Type-I and Type-II signals, which are found to be related to different parts/bands of the outer retina. Various example embodiments also characterized the light-induced optoretinography response of the outer retina under scotopic and photopic conditions, providing new angles to understand the physiological origin of the optoretinography signal. Besides, various example embodiments demonstrated enface mapping of the optoretinography signals in a wide field of view of 12°, analogous to the multifocal electroretinogram but with a much higher resolution, revealing the spatial distribution of the outer retina function. The approach according to various example embodiments can be widely applied to study the tissue-specific dynamics in a variety of animal models, as well as having great potential in investigating visual sensory pathways.

Accordingly, as an illustrative example, various example embodiments provide an automatic, unbiased approach for studying outer retina dynamics in response to visual stimuli in rodents. In various example embodiments, the method analyzes the comprehensive phase signals obtained from all the pixels in the speckle patterns (e.g., in a speckle layer, which may also be referred to as a target layer herein) in OCT images using a phase-sensitive spectral-domain OCT system. Various example embodiments also employ a phase-restoring subpixel motion correction method to correct the bulk tissue motion that degrades the accuracy of the phase measurement. The temporal phase signals at individual pixels may be projected into the feature space (e.g., principal component (PC) space) and then automatically classify them using hierarchical clustering. For example, to ensure consistency across data from different animals, a support vector machine (SVM) was trained in the established PC space using the labels obtained from the hierarchical clustering analysis. All subsequent analysis on different animals may thereafter be conducted by the trained SVM. The repeatability of the method was validated and the nanoscopic dynamics of the outer retina was studied under scotopic and photopic visual stimulation.

Accordingly, a need exists to automatically extract and classify the temporal phase signals extracted from individual pixels of OCT images to detect the micrometer-scale movements or nanometer-scale movements of various different outer retinal tissue types in response to a visual stimulus. In this regard, various example embodiments provide a method of performing optoretinography using OCT which automatically extracts the dynamics of outer retinal tissues in response to a visual stimulus. In various example embodiments, the method adopts unsupervised machine learning technologies to cluster temporal optoretinography signals (or more particularly, temporal phase signal) in a feature space (e.g., temporal or spatiotemporal feature space), which differentiates the response from photoreceptors from other outer retinal tissues, such as microvilli, RPE and BrM. Without the need for human intervention or prior knowledge of tissue distribution, the method according to various example embodiments can be extended to hunt for heterogeneous tissue responses to other external excitations, such as electrical and photothermal stimulation. In addition, in various example embodiments, a machine learning model (e.g., a classifier) is trained using the labels (labelled feature points) obtained, which enables the classification of new temporal phase signals based on the same criterion.

As explained hereinbefore, due to the limited optical resolution provided by OCT and ocular aberration, the outer retina typically exhibits speckle patterns in OCT images. To reveal the photoreceptor dynamics, some previous studies use adaptive optics to resolve individual photoreceptors. However, various example embodiments note that adaptive optics has a small field of view, limiting its potential applicability to clinics. Another conventional strategy is to ensemble average the signals from a selected region of interest, which may mix the heterogeneous responses from different retinal tissue types. In contrast, the method of performing optoretinography using OCT according to various example embodiments is able to reveal comprehensive nanoscopic dynamics from speckle patterns using a machine learning aided pipeline.

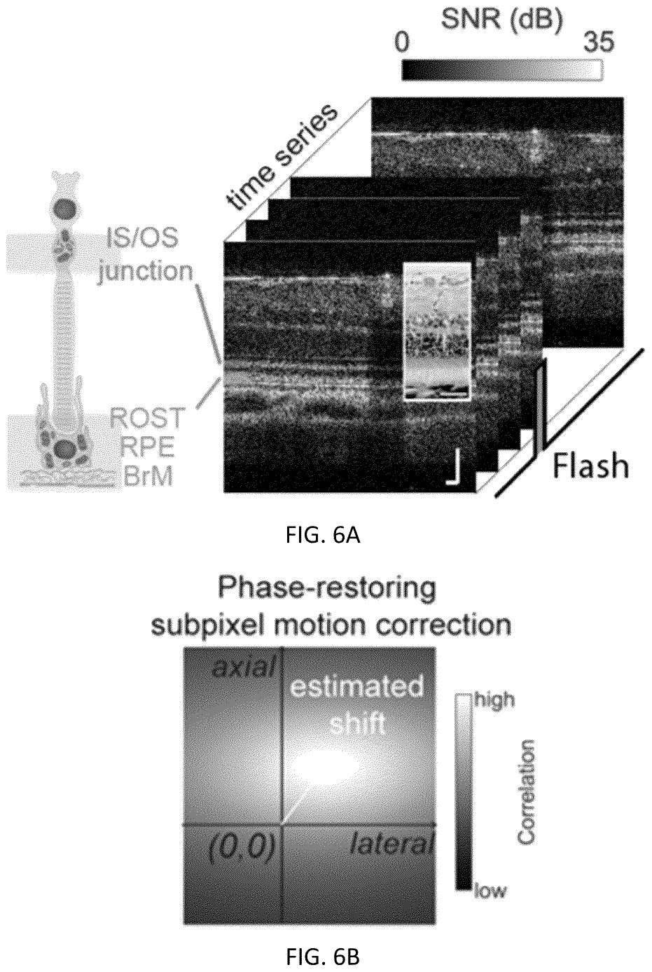

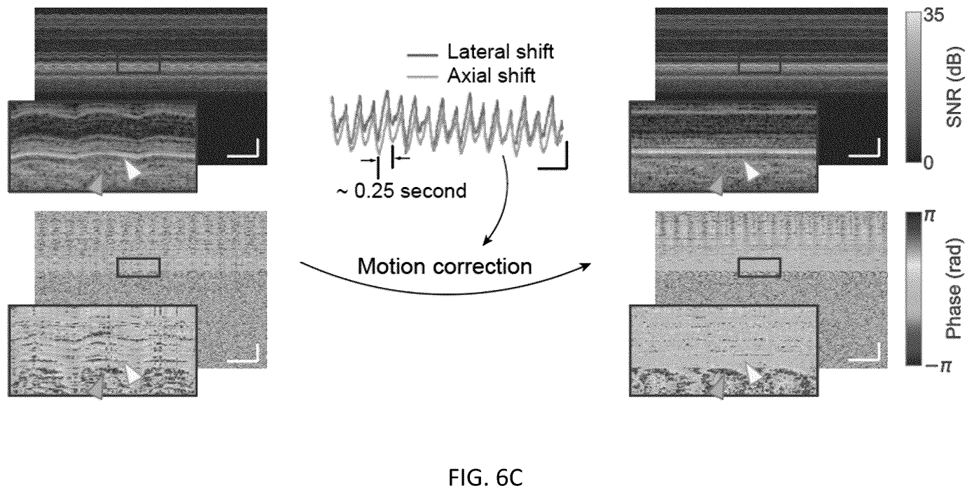

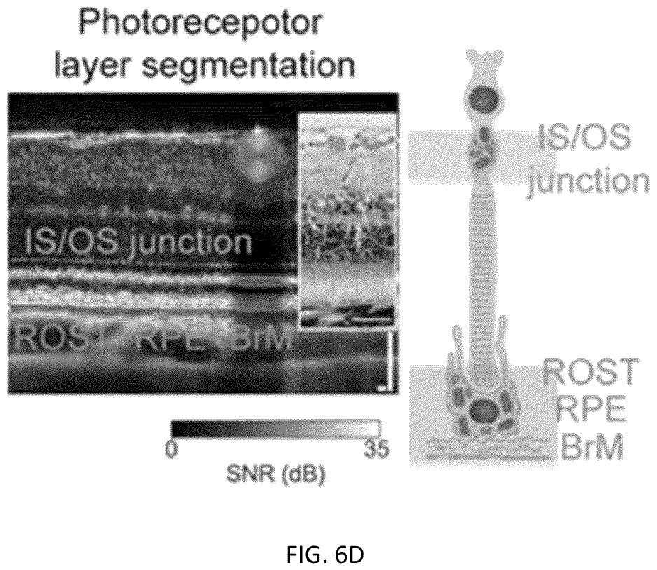

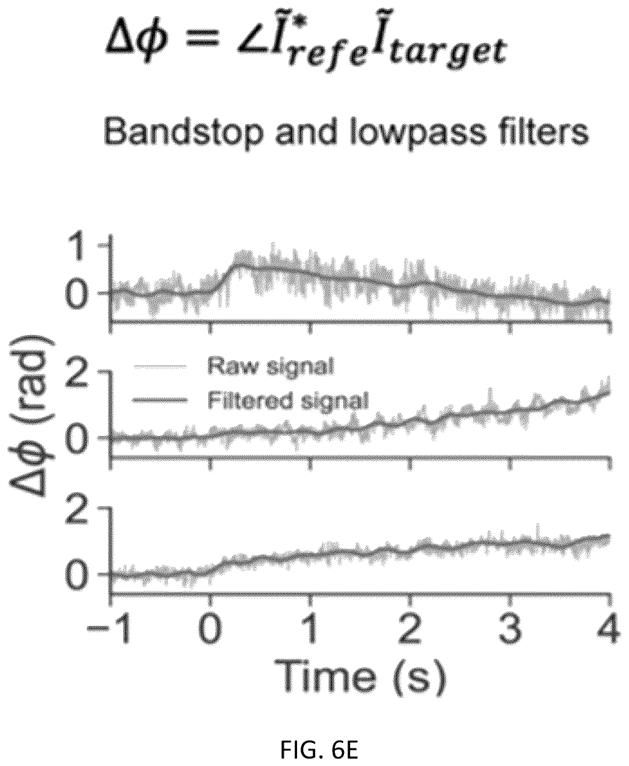

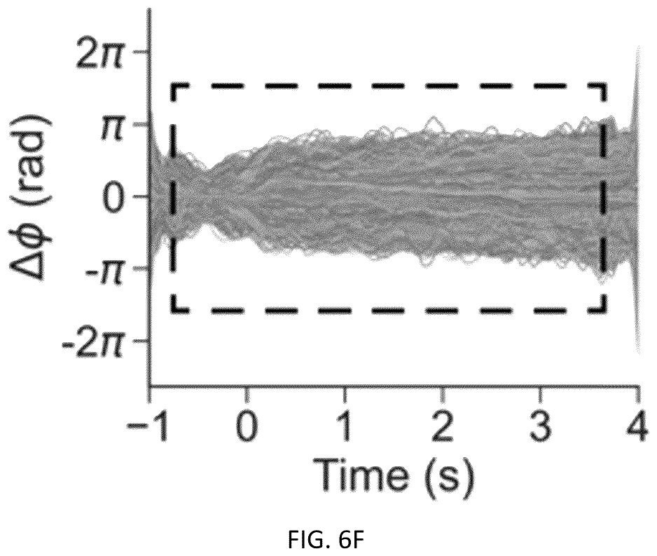

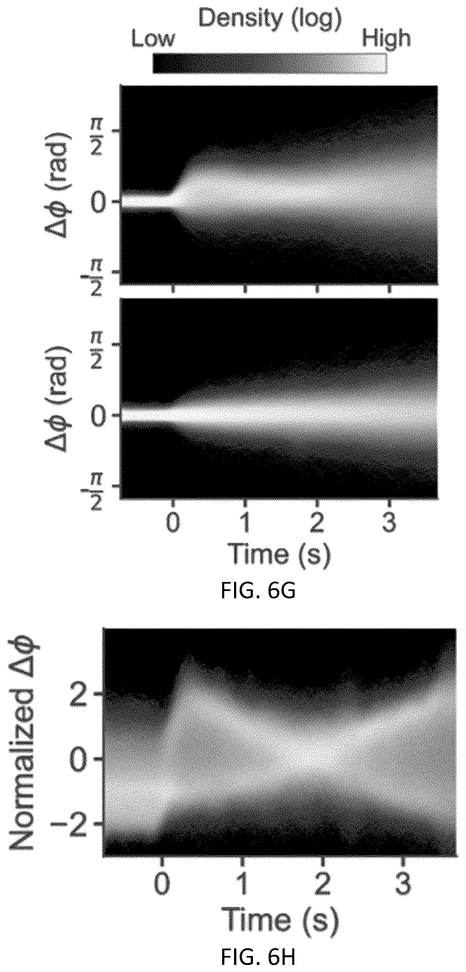

A method of training a machine learning model for classifying a temporal phase signal obtained from OCT images produced for optoretinography (e.g., corresponding to the method 100 of training a machine learning model as described hereinbefore with reference to FIG. 1) will now be described according to various example embodiments of the present invention. In particular, according to various example embodiments, phase responses (temporal phase signals, which may also be referred to as phase traces) are extracted from the outer retina. As an illustrative example, FIGS. 6A to 6I illustrate example image and signal processing pipeline/framework for the method of training a machine learning model according to various example embodiments of the present invention. Accordingly, various example embodiments provide a method for automatically extracting temporal optoretinography signals (or more particularly, temporal phase signals) in phase-sensitive OCT, preprocessing the extracted temporal phase signals, feature extraction, unsupervised clustering and training a machine learning model (e.g., a classifier).

In various example embodiments, the raw interferometric fringe in OCT images may first undergo standard OCT post-processing steps, including spectral calibration, k-linearity, dispersion compensation, and discrete Fourier transform (DFT), to obtain complex-valued OCT images. In this regard, the DFT of the processed spectral signals reconstructs the depth-resolved complex-valued OCT images.

The processing of OCT images and phase signals of pixels thereof will now be described according to various example embodiments of the present invention.

To suppress the influence of bulk tissue motion on the phase-sensitive measurement, subpixel-level translational displacements between repeated cross-sectional scans (B-scans) at the same location may be estimated using a single-step DFT algorithm. Then, the complex-valued OCT images may be registered using a phase-restoring subpixel motion correction approach, where the lateral and axial displacements are corrected by multiplying exponential terms in the spatial frequency domain and the spectrum domain, respectively.

In various example embodiments, in phase-sensitive optoretinography measurements, various example embodiments determine or calculate the temporal phase change (which may also be referred to as temporal phase difference) of a target layer with respect to a reference layer. In this regard, the light-evoked dynamics of the outer retina may be assessed by computing the temporal phase difference between two highly reflective outer retinal bands, namely, one from the reference layer (e.g., IS/OS junction) and the other from the target layer (which may be referred to as the complex layer or the mixed layer) comprising multiple outer retinal bands, such as corresponding to photoreceptor outer segment, microvilli, retinal pigment epithelium (RPE) and/or Bruch's membrane (BrM). Accordingly, the dynamics of the outer retina may be assessed by computing/extracting its temporal optical length (OPL) change by taking the IS/OS as a reference.

In various example embodiments, the reference layer and the target layer in the OCT images obtained may be automatically segmented using an automated image segmentation algorithm, such as based on graph theory and dynamic programming, or may be segmented manually. In various example embodiments, to improve the signal-to-noise ratio of motion detection, for each pixel in the target layer, a reference pixel or a reference pixel region (comprising a plurality of reference pixels) may be selected from the reference layer (e.g., IS/OS junction) centered at the same A-line as the pixel in the target layer. For example, each reference pixel region may comprise 5 adjacent A-lines (e.g., about 7.1 μm laterally (total width of the 5 adjacent A-lines)).

In the illustrative example, FIG. 6A shows example temporal serial cross-sectional OCT images (a series of OCT images) acquired from a 12° field of view on a rat's retina (more specifically, a series of repeated cross-sectional scans (or B-scans) obtained from the same location). In the outer retina, one hyperreflective band corresponds to the IS/OS junction, while the other hyperreflective band is a complex layer (which may also be referred to as a speckled layer or a target layer herein) comprising, for example, the outer segment tips of photoreceptors, microvilli, RPE and BrM. As shown in FIG. 6A, the OCT structural image agreed well with the histology (inset) (in FIG. 6A, the horizontal and vertical scale bars shown each corresponds to 100 μm). Accordingly, a series of OCT images produced by a phase-sensitive OCT system for optoretinography with respect to an outer retina may be obtained from a same location thereof, including a stimulus sub-series of OCT images produced in response to a visual stimulus to the outer retina (e.g., the flash illustrated in FIG. 6A). Each OCT image of the series of OCT images has a reference layer and a target layer (comprising a plurality of outer retinal bands), and in this regard, as described above, the same reference layer and the same target layer in each OCT image may be automatically segmented using an automated image segmentation algorithm or may be segmented manually.

For each OCT image of the series of OCT images and for each of a plurality of individual pixels in the target layer of the OCT image, a phase signal of the individual pixel in the target layer of the OCT image with respect to the reference layer of the OCT image may be determined to obtain, for each series of corresponding individual pixels of a plurality of series of corresponding individual pixels in the target layer across the series of OCT images, a temporal phase signal of the series of corresponding individual pixels. This results in a set of temporal phase signals for the plurality of series of corresponding individual pixels. In various example embodiments, the plurality of series of corresponding individual pixels covers all (or substantially all) pixels in the target layer across the series of OCT images (i.e., each pixel of the target layer in each OCT image). In various example embodiments, a series of corresponding individual pixels in the target layer across the series of OCT images refers to a series of one corresponding pixel in the target layer of each OCT image of the series of OCT images (e.g., corresponding to the same portion of the target layer across the series of OCT images). Various example embodiments may then correct or cancel out the systematic phase drift by self-referencing and remove arbitrary phase offset of the individual phase trace (individual temporal phase signal) by referring to its pre-stimulus frames.

In various example embodiments, the above-mentioned determining the phase signal of the individual pixel in the target layer of the OCT image comprises correcting a systematic phase drift of a complex-valued OCT signal of the individual pixel based on self-referencing to an individual pixel of the reference layer of the OCT image to obtain a first complex-valued OCT signal (which may be referred to as a pairwise self-referenced complex-valued OCT signal) of the individual pixel. In this regard, in various example embodiments, the systematic phase drift may be corrected (e.g., canceled out) by calculating the multiplication of the complex-valued OCT signal of the individual pixel (pixel of interest) in the target layer of the OCT image and the complex conjugate of the complex-valued OCT signal of the individual pixel in the reference layer of the OCT image, which may be defined by the following example expression:

$${\tilde{I}}_{tar / ref} ( i ) = {\tilde{I}}_{tar} ( i ) {\tilde{I}}_{r e f}^{*} ( i ) \qquad ( Equation 1 )$$

where Ĩₜₐᵣ(i) denotes the complex-valued OCT signal of the pixel of interest in the target layer, i denotes the index of the frame number, and Ĩ_{ref}(i) denotes the complex-valued OCT signal of the individual pixel in the reference layer. Ĩ_{tar/ref}(i) denotes the pairwise self-referenced complex-valued OCT signal (corresponding to the above-mentioned first complex-valued OCT signal) of the pixel of interest in the target layer. In Equation (1), ‘*’ represents complex conjugate.

In various example embodiments, to cancel out arbitrary phase offsets, each complex-valued phase trace (each individual phase signal) may be referenced to its pre-stimulus frames. In this regard, in various example embodiments, for each OCT image of the stimulus sub-series of OCT images, the above-mentioned determining the phase signal of the individual pixel in the target layer of the OCT image further comprises correcting a phase offset of the first (pairwise self-referenced) complex-valued OCT signal of the individual pixel based on a pre-stimulus sub-series of the series of OCT images produced prior to the visual stimulus to the outer retina to obtain a second complex-valued OCT signal (which may be referred to as a time referenced complex-valued OCT signal) of the individual pixel. In this regard, in various example embodiments, the phase offset of the first complex-valued OCT signal of the individual pixel may be corrected (e.g., cancelled) by determining an average of the first complex-valued OCT signals of corresponding individual pixels of the pre-stimulus sub-series of OCT images corresponding to the individual pixel of the OCT image to obtain an averaged pre-stimulus complex-valued OCT signal; and multiplying the first complex-valued OCT signal of the individual pixel with the averaged pre-stimulus complex-valued OCT signal to obtain the second complex-valued OCT signal of the individual pixel. Similarly, for each OCT image of the pre-stimulus sub-series of OCT images, the above-mentioned determining the phase signal of the individual pixel in the target layer of the OCT image may also comprises correcting a phase offset of the first complex-valued OCT signal of the individual pixel based on the pre-stimulus sub-series of the series of OCT images to obtain a second complex-valued OCT signal of the individual pixel and determining the phase signal of the individual pixel in the target layer of the OCT image in the same or similar manner as described above for each OCT image of the stimulus sub-series of OCT images. By way of an example only and without limitation, the above-described process for correcting a phase offset of the first (pairwise self-referenced) complex-valued OCT signal of the individual pixel may be defined by the following example expression:

$$Δ {\tilde{I}}_{tar / ref} ( i ) = {\tilde{I}}_{tar / ref} ( i ) \cdot \frac{1}{N} {\sum }_{i = 1}^{N} {\tilde{I}}_{tar / ref}^{*} ( i ) \qquad ( Equation 2 )$$

where ΔĨ_{tar/ref}(i) denotes time (pre-stimulus time) referenced signal (corresponding to the above-mentioned second complex-valued OCT signal), N represents the number of frames acquired before the light stimulus.

In various example embodiments, for each OCT image of the series of OCT images, the phase signal of the individual pixel in the target layer of the OCT image is determined based on the second complex-valued OCT signal of the individual pixel. As an example, the phase signal (which corresponds to the phase change/difference) of the individual pixel in the target layer of the OCT image may be extracted from the time reference signal according to the following example expression:

$$Δ ϕ ( i ) = ∠Δ {\tilde{I}}_{tar / ref} ( i ) \qquad ( Equation 3 )$$

where Δφ(i) denotes the phase signal extracted from the individual pixel (pixel of interest) in the target layer and ‘∠’ represents the calculation of argument. Accordingly, the temporal phase signal (which may also be referred to as the phase trace) of the series of corresponding individual pixels in the target layer across the series of OCT images may be obtained based on the phase signals of the series of corresponding individual pixels in the target layer across the series of OCT images.

In various example embodiments, as described hereinbefore, the above process described for determining the phase signal for one pixel of interest is respectively applied to every pixel in the target layer of each OCT image to extract the corresponding phase signal therefrom, to obtain a set of temporal phase signals for the plurality of series of corresponding individual pixels in the target layer across the series of OCT images.

In various example embodiments, the complex-valued OCT signal of the individual pixel in the target layer may be spatially averaged over multiple pixels in the reference layer (IS/OS junction) or the target layer or both to enhance the signal-to-noise ratio (SNR). In various example embodiments, the complex-valued OCT signal is spatially averaged over multiple pixels of a reference pixel region of the reference layer, and the correction (or cancellation) of the systematic phase draft and the correction (or removal) of the arbitrary phase offset may be performed as described below. That is, the phase signal of the individual pixel in the target layer of the OCT image may be determined with respect to a reference pixel region (comprising a plurality of individual pixels) of the reference layer of the OCT image.

In various example embodiments, in the case of determining the phase signal of the individual pixel in the target layer of the OCT image with respect to a reference pixel region of the reference layer of the OCT image, the systematic phase drift of the complex-valued OCT signal of the individual pixel may be corrected (e.g., canceled out) by calculating the multiplication of the complex-valued OCT signal of the individual pixel of interest in the target layer respectively with the complex conjugates of the complex-valued OCT signals of the individual pixels in the selected reference pixel region of the reference layer to obtain a set of first (pairwise self-referenced) complex-valued OCT signals of the individual pixel (each first complex-valued OCT signal paired with a respective reference pixel in the reference pixel region), which may be defined by the following example expression:

$${\tilde{I}}_{tar / ref} ( s , i ) = {\tilde{I}}_{tar} ( i ) {\tilde{I}}_{r e f}^{*} ( s , i ) \qquad ( Equation 4 )$$

where Ĩₜₐᵣ(i) denotes the complex-valued OCT signal of the pixel of interest in the target layer, i denotes the index of the frame number, Ĩ_{ref}(s,i) denotes complex-valued OCT signals of the individual pixels in the corresponding reference pixel region, and s denotes the pixel index in the reference region. Ĩ_{tar/ref}(s,i) denotes the pairwise self-referenced complex-valued OCT signals of the individual pixel, where the pixel of interest in the target layer is ergodically referred to all pixels (every pixel) in the reference pixel region. In Equation (4) above and Equation (5) below, ‘*’ represents complex conjugate.

In various example embodiments, to cancel out arbitrary phase offsets, each individual phase signal may be referenced to its pre-stimulus frames. In this regard, in various example embodiments, an average of the sets of first (pairwise self-referenced) complex-valued OCT signals of corresponding individual pixels of the pre-stimulus sub-series of OCT images corresponding to the individual pixel of the OCT image may be determined to obtain an averaged set of pre-stimulus complex-valued OCT signals; and the set of first complex-valued OCT signals of the individual pixel may then be multiplied with the averaged set of pre-stimulus complex-valued OCT signals to obtain a set of second (time (pre-stimulus time) referenced) complex-valued OCT signals of the individual pixel, which may be defined by the following example expression:

$$Δ {\tilde{I}}_{tar / ref} ( s , i ) = {\tilde{I}}_{tar / ref} ( s , i ) \cdot \frac{1}{N} {\sum }_{i = 1}^{N} {\tilde{I}}_{tar / ref}^{*} ( s , i ) \qquad ( Equation 5 )$$

where ΔĨ_{tar/ref}(s,i) denotes the set of time (pre-stimulus time) referenced complex-valued OCT signals of the individual pixel, N represents the number of frames acquired before the light stimulus.

The set of pre-stimulus time referenced complex-valued OCT signals may be subsequently averaged across the pixels in the reference region, and the phase information (phase signal) may be extracted from the averaged complex-valued OCT signal. In this regard, in various example embodiments, an average of the set of second complex-valued OCT signals of the individual pixel may be determined to obtain an averaged complex-valued OCT signal of the individual pixel; and the phase signal of the individual pixel in the target layer of the OCT image may be determined based on the averaged complex-valued OCT signal of the individual pixel, which may be defined by the following example expressions:

$$Δ {\tilde{I}}_{tar / ref} ( i ) = \frac{1}{S} {\sum }_{s = 1}^{S} Δ {\tilde{I}}_{tar / ref} ( s , i ) \qquad ( Equation 6 )$$

$$Δϕ ( i ) = ∠ Δ {\tilde{I}}_{tar / ref} ( i ) \qquad ( Equation 7 )$$

where Δφ(i) denotes the phase signal extracted from the individual pixel of interest in the target layer, ‘S’ denotes the total pixel number in the reference pixel region and ‘∠’ represents the calculation of argument. Therefore, the phase signal (which corresponds to the phase change/difference) of the individual pixel in the target layer of the OCT image may be extracted from the averaged complex-valued OCT signal according to Equation (7). Accordingly, the temporal phase signal (which may also be referred to as the phase trace) of the series of corresponding individual pixels in the target layer across the series of OCT images may be obtained based on the phase signals of the series of corresponding individual pixels in the target layer across the series of OCT images.

In various example embodiments, the temporal phase signal may then be converted into the OPL change (ΔOPL) by:

$$Δ OPL = \frac{{λ}_{0}}{4 π} Δϕ \qquad ( Equation 8 )$$

$$or$$

$$Δ OPL = - \frac{{λ}_{0}}{4 π} Δ ϕ \qquad ( Equation 9 )$$

where λ₀ is the central wavelength of the OCT system. In various example embodiments, then the reference layer is anterior to the target layer, the OPL change may be calculated using Equation (8). Otherwise, it may be calculated using Equation (9). This ensured that an increase in OPL change (ΔOPL) consistently represents an expansion between the target layer and the reference layer.

In various example embodiments, as described hereinbefore, the above process described for determining the phase signal for one pixel of interest is respectively applied to every pixel in the target layer of each OCT image to extract the corresponding phase signal therefrom, to obtain a set of temporal phase signals for the plurality of series of corresponding individual pixels in the target layer across the series of OCT images. In various example embodiments, when averaging phase signals across different pixels, the calculations may be performed in the complex plane to avoid biases introduced by phase wrapping.

In the illustrative example, and following on from FIG. 6A, FIG. 6B illustrates an upsampled cross-correlation map computed between repeated cross-sectional scans (B-scans), where the location of its peak was found as the estimated displacement, for the phase-restoring subpixel motion correction. FIG. 6C shows the time-elapsed intensity (upper) and phase (lower) M-scans without the phase-restoring subpixel motion correction, when no light stimulus was delivered to the retina (scale bar: 500 ms (horizontal), 100 μm (vertical)), the subpixel-level bulk motion estimated (the estimated temporal bulk tissue motion) by locating the peak of the upsampled cross-correlation map between repeated B-scans (scale bar: 500 ms (horizontal), 5 μm (vertical)) and the corresponding time-elapsed intensity (upper) and phase (lower) M-scans after the phase-restoring subpixel motion correction (after registration) (scale bar: 500 ms (horizontal), 100 μm (vertical)). In FIG. 6C, white and grey arrows label loci of the outer retina and the choroid, respectively. FIG. 6D shows an OCT structural image illustrating the segmentation of two hyperreflective layers in the outer retina, one hyperreflective layer corresponding to the IS/OS junction and the other hyperreflective layer corresponding to a complex layer (e.g., corresponding to the target layer) comprising the outer segment tips of the photoreceptors, RPE and BrM. As shown in FIG. 6D, the OCT structural image agreed well with the histology (inset) (in FIG. 6D, the horizontal and vertical scale bars shown each corresponds to 100 μm).

In various example embodiments, the extracted optoretinography signals (more particularly, temporal phase signals, which may also be referred to as phase traces) may then be processed by bandpass filters to minimize the residual artifacts induced by heartbeat and breathing. Subsequently, a low-pass filter (e.g., with a cut-off frequency of 10 Hz) may be used to filter out high-frequency oscillations. Alternatively, the extracted temporal phase signals may be lowpass filtered, and the remaining periodic oscillations induced by heartbeat and breathing may be suppressed by bandpass filters. After filtering, temporal phase signals with high variations in the baseline may be treated as noise and excluded from further analysis. For example, both ends of the temporal phase signals exhibited high variance due to edge effects, so only the center section may be selected for further analysis. For example, the temporal phase signals with a baseline standard deviation larger than 60 mrad may be excluded. Individual temporal phase signals (individual signal traces) may then each be normalized by subtracting its mean value and then dividing by its standard deviation (SD) to remove the differences in amplitude.

In the illustrative example, following on from FIG. 6D, FIG. 6E illustrates example representative temporal phase signals before and after bandstop and low-pass filtering.

According to various example embodiments, to facilitate the analysis of high-dimensional temporal phase signals, the extracted temporal phase signals may be projected into a feature space (e.g., temporal or spatiotemporal feature space) established by either automated feature extraction methods or manually selected features. For example, methods such as principal component analysis (PCA), self-organizing map and autoencoder, may be used to compress the dataset into a lower-dimensional spatiotemporal feature space. Alternatively, representative features may also be manually selected, such as amplitude, peak time and so on, to construct the spatiotemporal feature space. As an illustrative example, feature extraction using the PCA will now be described according to various example embodiments of the present invention. In particular, the PCA is used to compress datasets into a lower-dimensional feature space to facilitate the analysis of high-dimensional temporal phase signals.

In various example embodiments, an outlier detection method may be implemented to avoid undesirable elongated clusters in the subsequent unsupervised clustering analysis. For example, a distance-based outlier detection method may be used to preclude outliers distributed in the low-density region in the common PC space. An example method is that, for each data point (feature point) in the feature space (common PC space), for example, the minimum radius that comprises 2% of the group size may be calculated. Then, any data points with radii larger than Q₃+(Q₃−Q₁)/5 may be treated (labelled) as outliers and excluded from clustering analysis, where Q₁ and Q₃ are the first and third quartiles of the calculated radii.

In the illustrative example, to account for the inter-subject difference, for example, normalized temporal phase signals extracted from five rats were combined to calculate the PC coefficients. For example, the top three PCs capturing a total variance of 83.1% were selected to constitute a common PC space. Comparatively, suppose each dataset is projected into its own unique PC space, the top three PCs account for 83.4% (1.5%) variance on average, which is only 0.3% higher than that in the common PC space, indicating great consistency among datasets. In the illustrative example, FIG. 6F shows the filtered temporal phase signals (phase traces) extracted from the five rats. Temporal phase signals outside the dashed rectangle suffered from edge effects and were excluded from further analysis. In the illustrative example, following on from FIG. 6F, FIG. 6G shows the distribution density of stable phase response with (top) and without (bottom) light stimulus. FIG. 6G shows the distribution density of the normalized temporal phase signals, where individual temporal phase signals in the top panel of FIG. 6G were subtracted by its mean values and divided by its standard deviations. Accordingly, the processing of temporal phase signals and extraction of temporal signatures using PCA has been described. In particular, FIG. 6G shows the distribution density of phase traces that exhibited low variance prior to the light stimulus (before 0 second). When a flash was delivered to the retina (at t=0 second), function-associated phase responses can be clearly observed (top plot in FIG. 6G), whereas without a light stimulus, phase traces were gradually decorrelated without any noticeable response pattern (bottom panel). FIG. 6H shows each phase trace in the top plot of FIG. 6G normalized by subtracting its mean value and then divided by its standard deviation (SD).

As the illustrative example, FIG. 6I shows high-dimensional phase traces projected into a temporal feature space, or more particularly, a PC space constituted by top three PCs, with outliers being removed using a density-based method. In particular, FIG. 6I shows the distribution of normalized phase traces in the common PC space comprising the top three principal components, with outliers removed using a density-based outlier detection method. In FIG. 6I, the heatmaps show the distribution density of remaining data points (grey dots) when projected into different feature planes.

Various unsupervised clustering techniques, such as k-means clustering, hierarchical clustering, density-based spatial clustering of applications with noise and so on may be used to explore the common patterns in the feature spaces. As an illustrative example, unsupervised clustering using a hierarchical clustering method or algorithm will now be described according to various example embodiments of the present invention. In various example embodiments, an agglomerative hierarchical clustering algorithm may be used in combination with the Ward criterion to group individual points in the common PC space. This technique first computes the Euclidean distance of each pair of points and iteratively merges similar subclusters (e.g., two similar subclusters) into larger clusters. Under the guidance of the Ward method/criterion or minimum variance method, each merger guarantees there is a minimum increase of total within-cluster variance.

After generating the agglomerative hierarchical cluster tree, various clustering results can be constructed by cutting off the dendrogram at different levels. There exist a number of methods and criteria to determine cluster number. In various example embodiments, the optimal cluster number may either be manually set or determined by some metrics such as silhouette method, gap statistic method. As an illustrative example, various example embodiments seek to find the optimal cluster number that maximizes the overall inter-cluster dissimilarity (the averaged dissimilarity) between the reconstructed phase traces. In particular, when the phase traces are grouped into k clusters, filtered phase traces may be retrieved and averaged within individual clusters to reconstruct the phase traces. In other words, the filtered phase traces may be retrieved and averaged across individual clusters for each clustering result. Then the pairwise Pearson's cross-correlation coefficient p may be calculated for all n_{c}=(k−1)k/2 combinations of the reconstructed phase traces (between any two reconstructed phase traces), where k is the cluster number. For example, the root-mean-square (RMS) error of those cross-correlation coefficients relative to 1 (perfect correlation) may be calculated by:

$${C}_{R M S} = \frac{{\sum }_{n = 1}^{n = {n}_{c}} {( {ρ}_{n} - 1 )}^{2}}{{n}_{c}} \qquad ( Equation 10 )$$

The cluster number with the largest C_{RMS} may be determined as the optimal cluster number. Accordingly, in various example embodiments, the largest inter-cluster difference is sought.

Accordingly, as described hereinbefore according to various example embodiments, temporal phase signals (phase traces) obtained may be projected to feature points in a feature space (e.g., temporal or spatiotemporal feature space). Furthermore, these feature points in the feature space may be labelled. In various example embodiments, the labelling of the feature points in the feature space may include grouping the plurality of feature points into a plurality of clusters in the feature space (e.g., using an unsupervised clustering technique); and labelling, for each of the plurality of clusters, feature points belonging to the cluster with a label assigned to the cluster. In various example embodiments, the plurality of clusters has a plurality of different labels assigned thereto, respectively. In this regard, the plurality of different labels comprising a plurality of different outer retinal band labels corresponding to the plurality of outer retinal bands of the target layer.

The training of a machine learning model (e.g., support vector machine (SVM) model) in the established/same feature space (e.g., the established/same PC space) will now be described according to various example embodiments of the present invention. To classify new temporal phase signals based on the same criterion, a machine learning model, for example, a classifier, such as SVM, Bayes classifier, neural network and so on, may be trained in the same feature space using the labelled feature points therein. In various example embodiments, each phase trace is projected onto the PC space which comprises the top three principal components. For each phase trace, its value along these three principal components or its coordinate in the principal component space may be referred to as a score in the PC space. Therefore, for training the machine learning model, the scores in the PC space are features and their labels were obtained by the above-mentioned unsupervised clustering. The objective is to assign each feature point (data point) in the feature space (in this example, a PC space) to a cluster. Accordingly, feature points are first labelled in the feature space using an unsupervised learning method, and then a machine learning model (e.g., a classifier) is trained in the same feature space using the labelled feature points.

For example, training a SVM can be implemented using the “templateSVM” and “fitcecoc” functions in MATLAB. The input features may be standardized before training, the gaussian kernel was selected, and automatic hyperparameters optimization was turned on. Subsequently, new temporal phase signals (extracted from new OCT images in the same or similar manner as described hereinbefore according to various example embodiments) can be classified using the trained classifier in the feature space. As an illustrative example, the decision boundaries of each cluster may be extracted by training a SVM in the common PC space with the previously obtained labels, including outlier, Type-I signal, and Type-II signal. Evaluated with the 10-fold cross-validation strategy, the trained SVM achieves a classification accuracy of 99.5%. Accordingly, the trained SVM is useful when processing a new dataset (new series of OCT images) with the same criterion. In this regard, the temporal phase signals (phase traces) extracted from the new dataset may be preprocessed and projected into the common PC space in the same or similar manner as described hereinbefore according to various example embodiments. For example, each new phase trace may be projected onto the feature space, such as the PC space according to the principal coefficients. In the PC space, each feature point (data point), which corresponds to a corresponding pixel in the target layer of the OCT images, may be assigned to a cluster using the trained SVM. Then, Type-I and Type-II signals can be reconstructed based on the classification results from the trained SVM.

Accordingly, in various example embodiments, there is provided a method of classifying a temporal phase signal obtained from OCT images produced for optoretinography using the trained machine learning model. The method comprises: obtaining a series of OCT images produced by a phase-sensitive OCT system for optoretinography with respect to an outer retina, including a stimulus sub-series of OCT images produced in response to a visual stimulus to the outer retina; determining, for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands; projecting the temporal phase signal of the series of corresponding individual pixels to a feature point in a feature space; and classifying the temporal phase signal using the trained machine learning model based on the feature point which the temporal phase signal has been projected to. In various example embodiments, the series of OCT images may be obtained, the phase signal of the individual pixel in the target layer of the OCT image may be determined, and the temporal phase signal of the series of corresponding individual pixels may be projected to a feature point in the feature space in the same or similar manner as described hereinbefore with respect to the training of the machine learning model according to various example embodiments. For example, a new temporal phase signal extracted from new OCT images of an outer retina may thus be classified using the trained machine learning model into one of a plurality of outer retinal tissue types corresponding to a plurality of outer retinal bands of the target layer or an outlier type.

For example, for repeated volumetric scans, in various example embodiments, the temporal phase signals (phase traces) may be extracted from the OCT images using the same method as for repeated cross-sectional scans (B-scans) as described hereinbefore according to various example embodiments. The phase traces may be thresholded, normalized and classified using a pretrained machine learning model (e.g., SVM) in the common downsampled PC (d-PC) space. A new machine learning model (e.g., SVM) may then be trained to classify the phase signals extracted from the volumetric scans. The previously extracted top three PCs may be down-sampled at the same time points as the repeated volumetric scans to constitute a common d-PC space. As an illustrative example, the raw phase signals extracted from five healthy rats using the repeated B-scans protocol were combined and down-sampled at the same time points as the repeated volumetric scans. The signals with a baseline standard deviation larger than 0.3 rad may be treated as decorrelated noise and may be removed from further analysis. Individual phase traces may then be normalized by subtracted its mean value, divided by its standard deviation. The normalized phase traces may be projected into the common d-PC space (to feature points in the common d-PC space), which serves as the features for SVM training. Their labels may be obtained by retrieving their corresponding complete phase traces, followed by preprocessing and classification by the previously trained SVM in the common PC space, except that there is no additional thresholding process. In an experiment, a new SVM was trained in the common d-PC space using the same implementation mentioned above, achieved a classification accuracy of 83.4% with the 10-fold cross-validation method.

In particular, as an illustrative example, FIGS. 6A to 6I illustrate the example image and signal processing pipeline/framework for optoretinography on wild-type rats for the method of training a machine learning model according to various example embodiments of the present invention, whereby the machine learning model was trained in a temporal feature space. In particular, the method of training a machine learning model for classifying a temporal phase signal obtained from OCT images was validated in optoretinogram experiments on wild-type rats and the corresponding example image and signal processing pipeline/framework are illustrated in FIGS. 6A to 6I.

FIG. 6A shows the serial cross-sectional scans (or B-scans, corresponding to a series of OCT images) acquired from a rat's retina at a same location. As shown in FIGS. 6B and 6C, a phase-restoring subpixel motion correction method was adopted to correct the bulk motion with subpixel precision. In this regard, the bulk tissue motion between repeated cross-sectional scans were estimated using a single-step DFT method, where the peak of the upsampled cross-correlation map was found as the estimated displacement. FIG. 6C also shows the time-elapsed intensity and phase maps extracted from a dataset comprising repeated cross-sectional scans (B-scans) at a same location without light stimulus, which manifests high stability in the photoreceptor layer. Then, the IS/OS and outer retina were automatically segmented based on the averaged B-scan, as illustrated in FIG. 6D.

The phase signals were extracted from each pixel in the outer retina (or more specifically, in the target layer) by taking the IS/OS as reference and processed by bandstop filters and a lowpass filter. Compared with raw phase traces (light gray lines in FIG. 6E), the periodic oscillations and high-frequency fluctuations were significantly suppressed after filtering (dark gray lines in FIG. 6E). FIG. 6F shows the filtered phase traces extracted from five rats, where both ends of the signals with high variations due to the edge effects were excluded from further analysis (signals outside of the dashed black rectangle in FIG. 6F were removed). Individual phase traces with low phase fluctuation in baseline were selected (see top plot of FIG. 6G) and normalized by subtracting its mean value and divided by its standard deviation (FIG. 6H). For example, a PCA method was then employed to extract the principal components (PCs).

Accordingly, in various example embodiments, agglomerative hierarchical clustering and machine learning (e.g., SVM) in the PC space enable automated, unbiased optoretinogram. To reveal the comprehensive dynamics of the outer retina in response to light stimulus in an unbiased manner, unsupervised learning was employed via agglomerative hierarchical clustering to group individual temporal phase signals (phase traces) into distinct types. A SVM model was then trained using the labels obtained in the clustering analysis and their features in a common PC space, thereby enabling the classification of temporal phase signals extracted from new animals with the same decision boundaries. In particular, the cross-sectional or volumetric scans shown in FIG. 6A were acquired in time sequence from a 12° field of view (FOV) on wild-type rats' retina. The phase difference between two hyperreflective bands in the outer retina was computed to reveal its dynamics. As illustrated in FIG. 6A, the upper hyperreflective layer corresponds to the IS/OS junction of the photoreceptors, whereas the lower hyperreflective layer corresponds to a target layer (which may also be referred to as a complex or mixed layer) comprising multiple types of tissues that existing imaging modality cannot resolve such as explained in the background. In various example embodiments, the target layer comprises the outer segment tips of the photoreceptors, microvilli, RPE, and BrM. Despite the high motion detection sensitivity of phase-sensitive OCT, its stability is susceptible to bulk tissue motion, which would result in apparent image distortions in the time-elapsed intensity map and destroy the phase stability (see FIG. 6C). To eliminate the negative influence of bulk tissue motion, a phase-restoring subpixel motion correction method was employed to register complex-valued OCT images down to subpixel precision. For example, as shown in FIG. 6C, it can be observed that the periodical oscillations on the order of a few micrometers in the estimated lateral and axial shifts matched the frequency of heart pulsation (about 4 Hz). After image registration, as shown in FIG. 6C, excellent motion stability was achieved at the photoreceptor layers (white arrows), while slight periodical oscillations could be observed in the choroid (grey arrows).

For each pixel in the target layer, its temporal phase change was computed with respect to the IS/OS layer. To account for inter-subject differences, temporal phase signals from five healthy rats were combined for the subsequent feature extraction and unsupervised clustering. The extracted temporal phase signals were filtered and thresholded in the baseline. When a flash was delivered to the retina, function-associated phase responses were clearly observed (see top plot in FIG. 6G). For comparison, without light stimulus, the phase signals were gradually decorrelated and there was no salient response pattern (see bottom plot in FIG. 6G). Individual phase traces in the top plot of FIG. 6G were then normalized by subtracting its mean value and then dividing by its standard deviation. The normalization process contrasts the two signal patterns in the distribution density map (see FIG. 6H). Then the complex temporal phase signals (phase traces) were featured by PCA to top three components to constitute a common PC space, as shown in FIG. 6I. In FIG. 6I, the outliers have been removed and the remaining data points (grey dots) are differentiated using a distance-based outlier detection algorithm to avoid undesirable elongated clusters in subsequent unsupervised clustering.

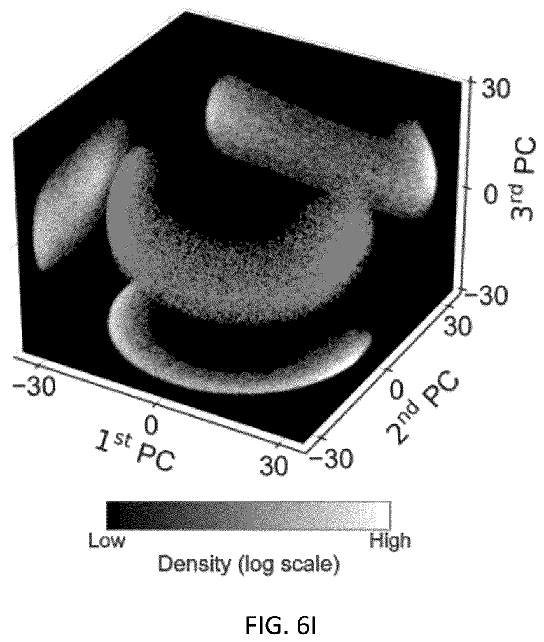

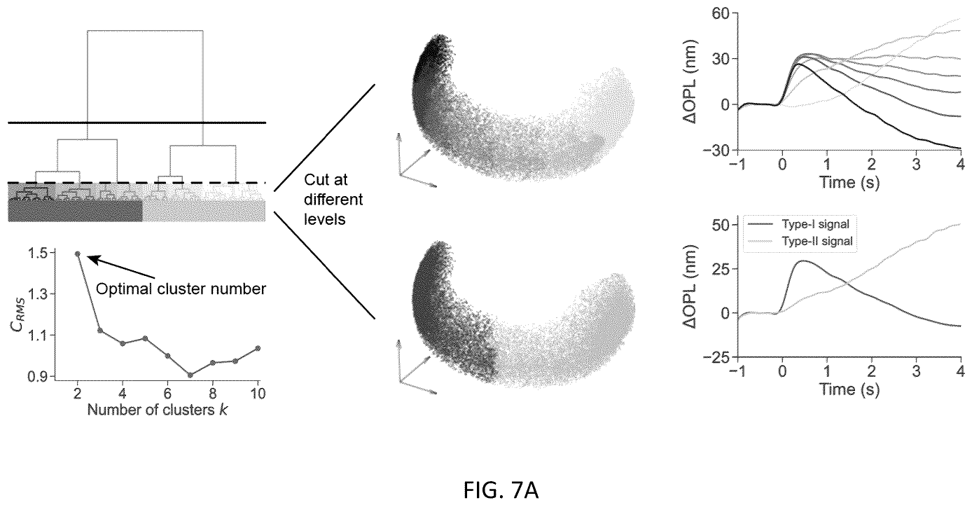

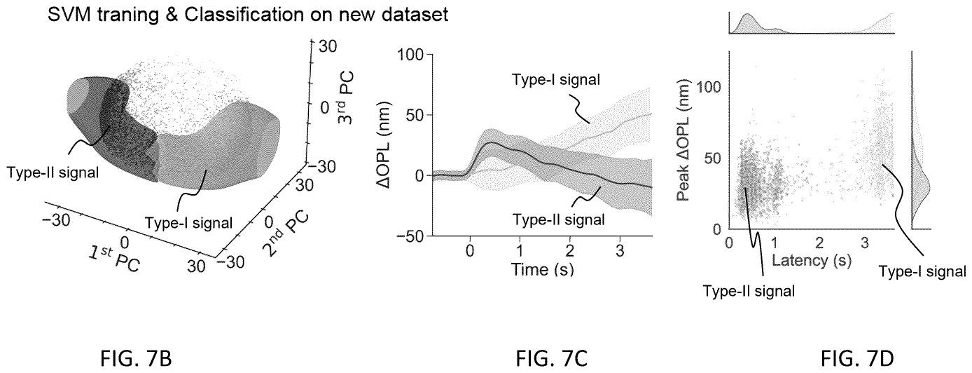

As shown in FIG. 7A, thresholding the dendrogram at different levels leads to different clustering results. For example, FIG. 7A shows the clustering results when signals were grouped into 7 clusters by cutting off the hierarchical tree along the dashed line. Individual temporal phase signals (phase traces) were retrieved and averaged within each cluster. In particular, FIG. 7A illustrates the unsupervised clustering using hierarchical clustering and subsequent classification on new dataset using the trained SVM, including: a dendrogram showing the cluster structure of the signals in the common PC space, whereby only top 100 subclusters are shown; clustering signals in the PC space into 2 clusters by cutting the dendrogram along the solid black line along with the corresponding averaged reconstructed Type-I and Type-II signals (Type-I and Type-II signals were obtained by retrieving temporal phase signals and averaging across each cluster); C_{RMS} value with respect to different cluster numbers (the optimal cluster number was determined by the cluster numbers corresponding to the maximum C_{RMS} value); clustering signals in the PC space into 7 clusters by cutting the dendrogram along the dashed black line along with the corresponding averaged reconstructed signals for each cluster.

In various example embodiments, to obtain the optimal cluster number, C_{R}MS was calculated to quantify the averaged dissimilarity between reconstructed signals, where a larger C_{RMS} value represents a larger inter-cluster dissimilarity. As shown in FIG. 7A, C_{RMS} reached maximum when the cluster number equals to 2. Accordingly, signals were grouped into 2 clusters by cutting off the clustering tree along the solid black line in FIG. 7A. FIG. 7A also shows the corresponding clustering results and reconstructed signals. The first type signal (Type-I signal) manifests rapid elongation followed by a gradual recovery, while the second type of signal (Type-II signal) exhibits a slow elongation.

Accordingly, to automatically classify the temporal phase signals (phase traces) in the common PC space (e.g., grey dots in FIG. 6I), various example embodiments may use an agglomerative hierarchical clustering algorithm based on Ward's criterion. Thresholding the dendrogram at different levels leads to different clusters (see FIG. 7A). As an example, FIG. 7A shows a clustering result in the PC space when signals were divided into 7 groups along the dashed black line shown in the dendrogram. The corresponding reconstructed phase signals may be averaged within a corresponding cluster and converted into optical path length change (ΔOPL). For example, as shown in FIG. 7A, two salient types (Type-I and Type-II signals) and some intermediate types were detected. To determine the optimal cluster number for further quantitative analysis, various example embodiments may calculate the inter-cluster correlation coefficients between averaged reconstructed phase signals and estimated their root mean square (RMS) relative to 1 (perfect correlation), which is termed C_{RMS}. In this regard, a higher C_{RMS} indicates a larger inter-cluster dissimilarity. In the illustrative example, as shown in FIG. 7A, when the cluster number is 2, C_{RMS} reaches its maximum to best distinguish the reconstructed phase traces between clusters. Therefore, the dendrogram may be thresholded along the solid black line shown in FIG. 7A to divide the signals in the PC space into 2 clusters. The corresponding reconstructed signals were averaged within the clusters, the first type of signal (Type-I signal) exhibits a biphasic trend with a rapid elongation followed by a gradual recovery, while the second type of signal (Type-II signal) shows a slow elongation.

In various example embodiments, to classify new signals with the same criterion, a SVM model may then be trained using the labels obtained to set the boundaries for Type-I signals and Type-II signals in the common PC spaces as shown in FIG. 7B. Using such a trained SVM, temporal phase signals extracted from new OCT image datasets can be preprocessed and projected into the same established PC space. These new temporal phase signals (phase traces) were successfully classified into Type-I signals, Type-II signals, and outliers, according to the trained SVM classification boundaries. FIG. 7C shows the average phase traces of the classified Type-I signals and Type-II signals, with the bands showing their standard deviations. As shown in FIG. 7D, the peaks and theirs corresponding latencies of ΔOPL from individual Type-I and Type-II signals exhibited distinct distributions. In particular, FIG. 7C shows the corresponding reconstructed temporal phase signals. The peak ΔOPL and latency of individual temporal phase signals (phase traces) are plotted in FIG. 7D, where Type-I and Type-II signals exhibited distinct distributions.

Accordingly, FIG. 7B shows the trained SVM decision boundaries for Type-I signal and Type-II signal in the PC space. In FIG. 7B, dots represent the phase traces extracted from the new OCT image dataset and after being preprocessed and projected into the same PC space. They were classified into Type-I signal, Type-II signal and outliers using the trained SVM. FIG. 7C shows the reconstructed Type-I signal and Type-II signal corresponding to dots in FIG. 7B according to the classification result in FIG. 7B. In FIG. 7C, the solid lines and bands denote the averaged value and the range of standard deviation (±standard deviation), respectively. FIG. 7D shows the distribution of the peak ΔOPL and corresponding latency of individual signals in FIG. 7C.

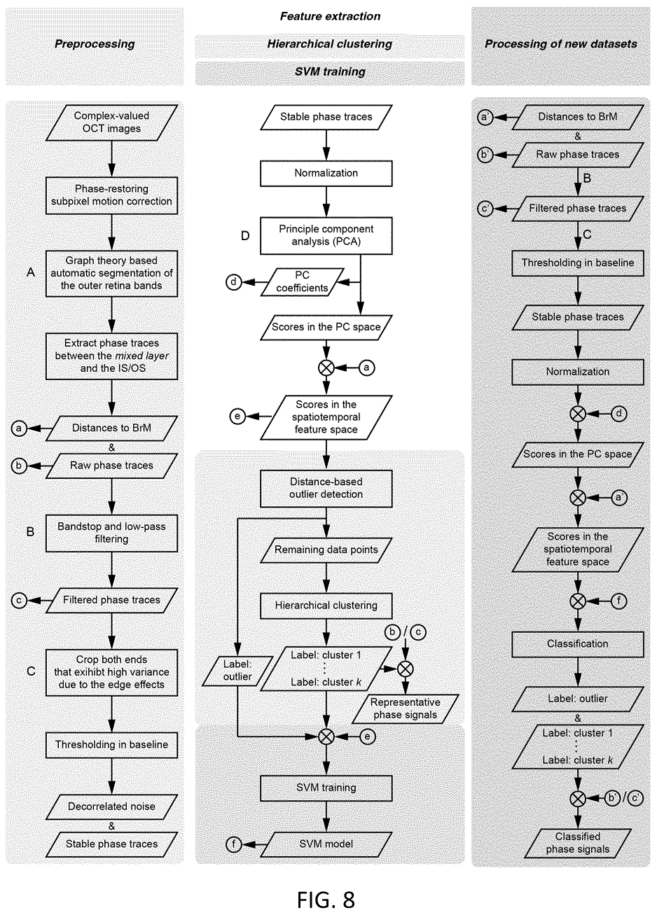

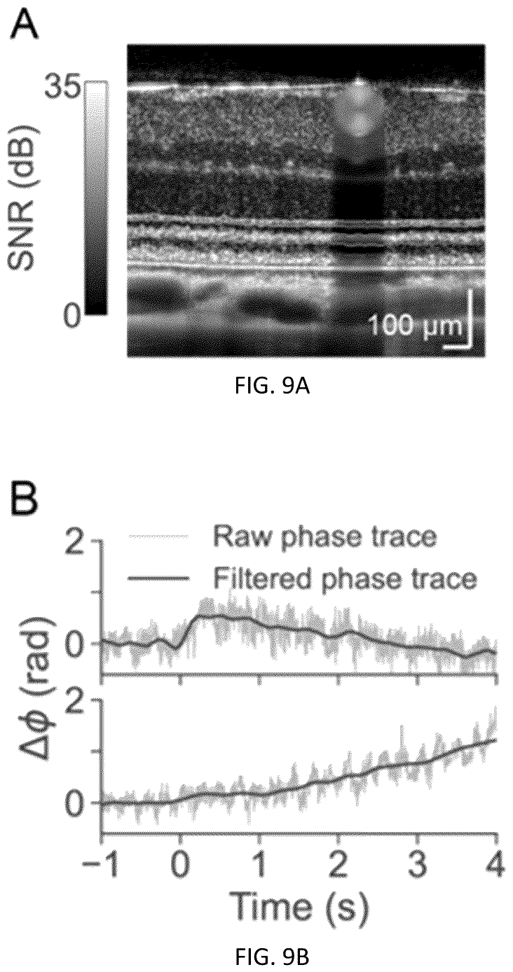

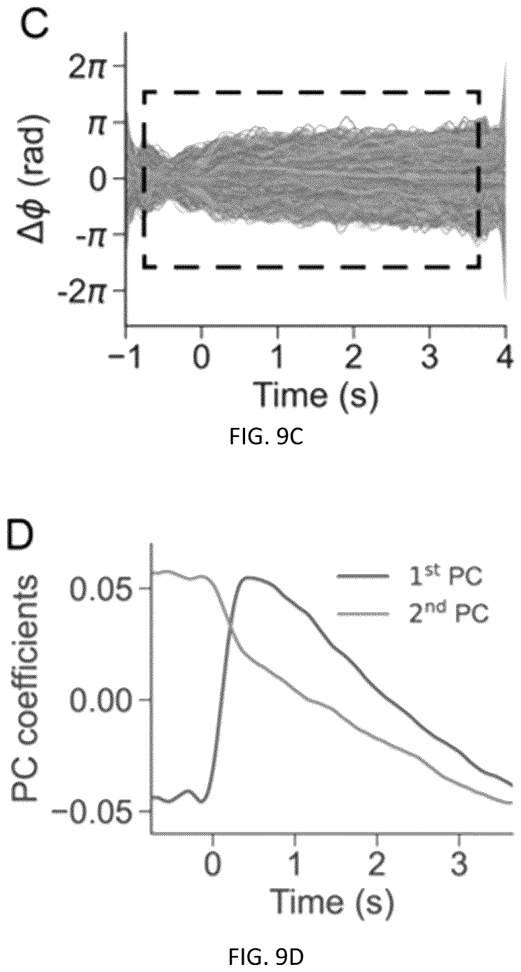

FIG. 8 depicts a flow chart showing an overview of the processing complex-valued OCT signals of OCT images to obtain (extract) temporal phase signals for training a machine learning model (e.g., a SVM model), the training of the machine learning model and the classification of new temporal phase signals obtained with respect to a target layer (mixed layer) using the trained machine learning model, according to various example embodiments of the present invention. In various example embodiments, the machine learning model may be trained in a temporal feature space or a spatiotemporal feature space. In this regard, as an illustrative example, FIG. 8 shows the machine learning model trained in a spatiotemporal feature space. For example, as shown in FIG. 8, phase traces extracted from the mixed layer are preprocessed and projected onto the spatiotemporal feature space. Distinct phase responses (phase traces) are identified using hierarchical clustering. A SVM may then be subsequently trained in the same spatiotemporal feature space to facilitate the classification of new phase traces with the same criterion. For better understanding, FIGS. 9A to 9D illustrate various stages (labelled with ‘A’, ‘B’, ‘C’ and ‘D’) of the flow chart shown in FIG. 8. Further experimental results will now be described or discussed according to various example embodiments of the present invention.

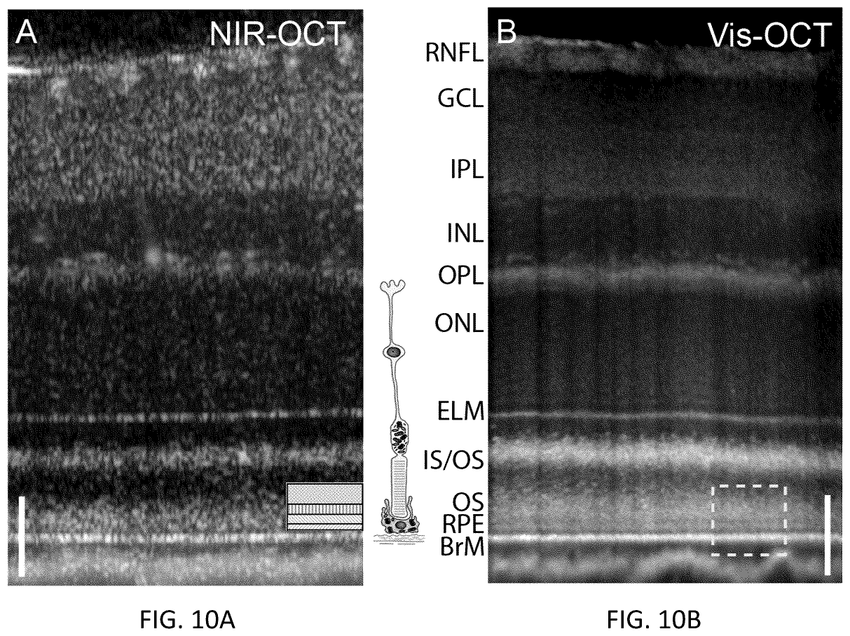

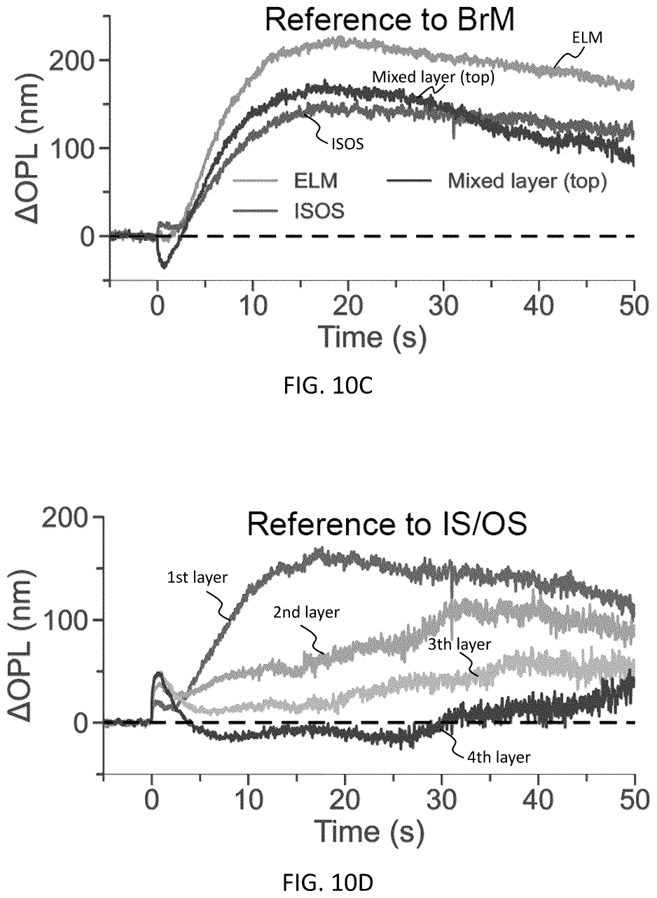

FIGS. 10A to 10D show retinal layers and their dynamics in response to visual stimuli in experiments conducted. FIG. 10A shows an averaged retinal B-scan from NIR-OCT. FIG. 10B illustrates the ultrahigh-resolution vis-OCT image which helps to distinguish photoreceptors' OS, RPE, and BrM layers. An enlarged view of the dashed box is shown in FIG. 10B (scale bar=50 μm). NIR-OCT was used for optoretinography imaging experiments. FIG. 10C shows the optoretinography signals obtained from various hyperreflective bands in the outer retina by taking the BrM as the reference. FIG. 10D shows the optoretinography signals obtained at various depths in the mixed layer (target layer) relative to IS/OS, corresponding to patterned bars shown at the bottom right of FIG. 10A. Herein, RNFL denotes retinal nerve fiber layer; GCL denotes ganglion cell layer; IPL denotes inner plexiform layer; INL denotes inner nuclear layer; OPL denotes outer plexiform layer; ONL denotes outer nuclear layer; ELM denotes external limiting membrane; IS/OS denotes inner segment/outer segment junction; OS denotes outer segment; RPE denotes retinal pigment epithelium; BrM denotes Bruch's membrane.

In particular, in relation to FIGS. 10A to 10D, all optoretinography imaging experiments were performed in-vivo using a custom-built NIR-OCT with an axial resolution of 2.0 μm in tissue. Cross-sectional (see FIG. 10A) and volumetric scans were acquired in time sequences from a 120 field of view (FOV) on wild-type rat retinas. A custom-built ultra high-resolution visible-light OCT (vis-OCT), which adopted the same optical design as the NIR-OCT but provided 1.1 μm axial resolution in tissue, was used to validate retinal layer delineation (see FIG. 10B). As illustrated in FIGS. 10A and 10B, several hyperreflective layers were observed in the outer retina, including the external limiting membrane (ELM), the IS/OS junction, BrM and a thick speckling layer between the IS/OS and the BrM, which may be referred to herein as the target or mixed layer. As verified by the vis-OCT, the mixed layer comprises photoreceptors' OS and RPE cells that the conventional NIR-OCT system cannot resolve. To reveal the light-evoked dynamics in the outer retina, according to various example embodiments, the changes in the optical path length (OPL) (phase difference) was first computed between the BrM and three hyperreflective bands in the outer retina, including ELM, IS/OS, and the top band of the mixed layer (the most top patterned bar shown in the bottom right of FIG. 10A). To achieve high phase stability/sensitivity in-vivo, according to various example embodiments, the bulk tissue motion is corrected. For example, a phase-restoring motion correction method to register complex-valued OCT images with subpixel precision (e.g., such as described in H. Li, et al., “Shot-noise limited phase-sensitive imaging of moving samples by phase-restoring subpixel motion correction in Fourier-domain optical coherence tomography”, bioRxiv, June 2022) may be employed. Phase traces extracted from the extended optoretinography experiments (55 seconds in duration) were spatially averaged across pixels within each band. In various example embodiments, when converting the phase change into the OPL change, an additional negative sign was added if the target layer was anterior to the reference layer. This ensured that an increase in OPL always represents an expansion between the two layers.

As shown in FIG. 10C, distance between ELM and BrM increases after the stimulus (1 ms, 500 nm, 0.18% bleach level) at a rate of about 26 nm/s, reaching 220 nm around 20 seconds, and then slowly recovers afterwards. The IS/OS layer rapidly (about 1 second) moves away from BrM, bounces back within 2 seconds and then continues to slowly move away from the BrM. The top band (1st layer) of the mixed layer (presumably OS tips) rapidly (about 1 second) moves toward the BrM by about 35 nm, then moves away from it over the next 20 seconds, followed by a slower recovery afterwards.

Conventionally, optoretinography monitors the movement of OS tips relative to the IS/OS layer. Due to the ambiguity of multiple tissue layers in NIR-OCT, various example embodiments recorded the OPL changes from several outer retinal bands in the mixed layer, indicated by various patterned bars at the bottom right of FIG. 10A, relative to the IS/OS. As shown in FIG. 10D, the OPL changes in different bands comprises a mixture of two distinct signatures: (a) rapid (about 1 second) expansion followed by a slower (about 10 seconds) decline, and (b) slow (about 20 second) expansion, followed by even slower recovery. Accordingly, various example embodiments address a technical problem of the superposition of distinct signals by performing signal decomposition and classification to determine the tissue origin associated with each individual temporal phase signal.

Unsupervised learning of spatiotemporal patterns for signal classification will now be described according to various example embodiments of the present invention. The axial resolution limit of NIR-OCT leads to the mixing of light-evoked responses from both OS and RPE, thereby complicating the interpretation of the results. To identify distinct signal patterns in an unbiased manner and group them into distinct types, individual phase traces were projected onto a spatiotemporal feature space and unsupervised learning was employed using agglomerative hierarchical clustering based on Ward's criterion. A SVM model was then trained on these type labels within the established feature space, enabling the extraction of each cluster's decision boundary and the classification of new phase traces using the same decision boundaries.

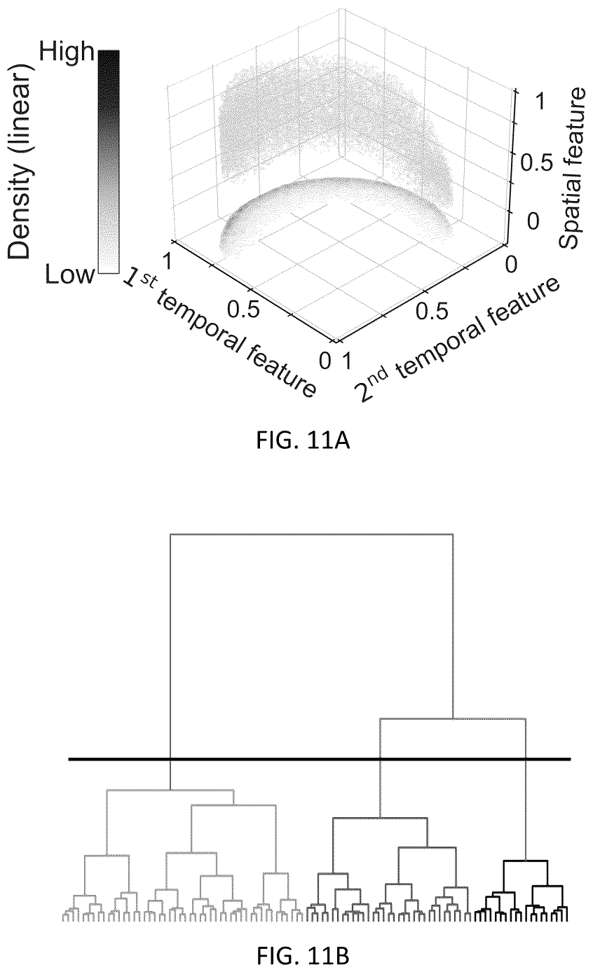

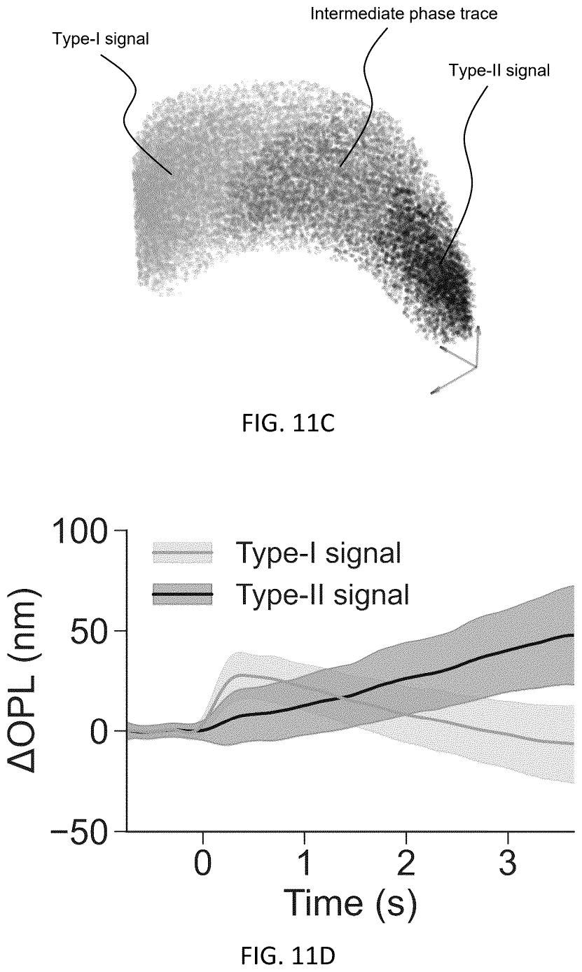

FIGS. 11A to 11D illustrate the unsupervised clustering in the spatiotemporal feature space for signal classification according to various example embodiments of the present invention. In this regard, the machine learning model is trained in the spatiotemporal feature space FIG. 11A shows the distribution of the signals in the 3D spatiotemporal feature space. The outliers have been removed using a distance-based detection method, while the remaining data points (feature points) are shown. The heatmap shows the distribution density of the remaining data points when projected onto the temporal feature plane. FIG. 11B depicts a dendrogram showing the cluster structure of the remaining phase traces (corresponding to the dots in FIG. 11A) in the spatiotemporal feature space, with only the top 100 subclusters displayed. FIG. 11C shows the clustering of the remaining phase traces in the spatiotemporal feature space into three clusters by thresholding the dendrogram along the solid black line shown in FIG. 11B. Dots with different shades of grey correspond to different groups. FIG. 11D shows the corresponding representative Type-I and Type-II signals obtained by averaging the individual phase traces within each cluster.

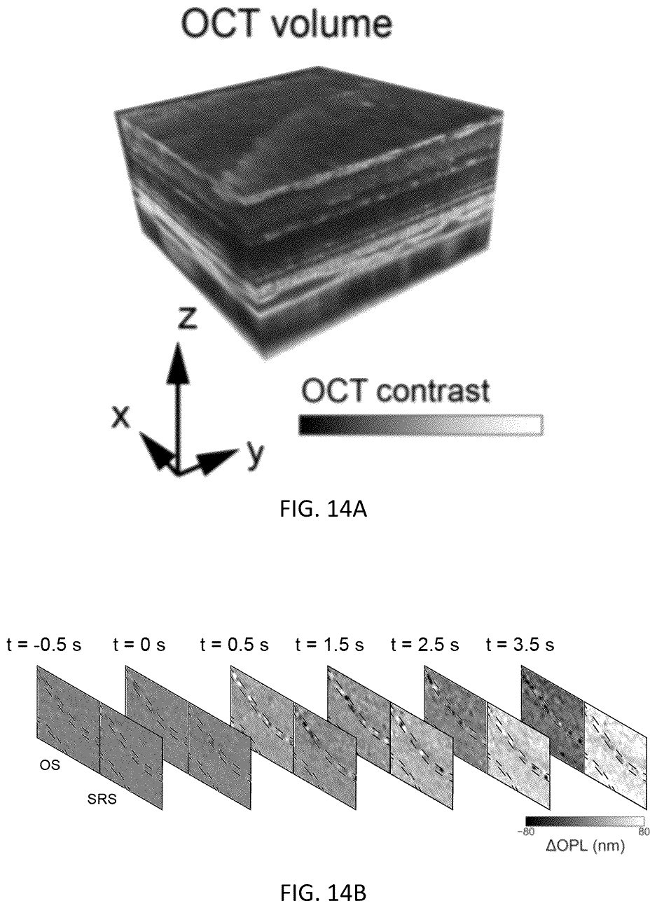

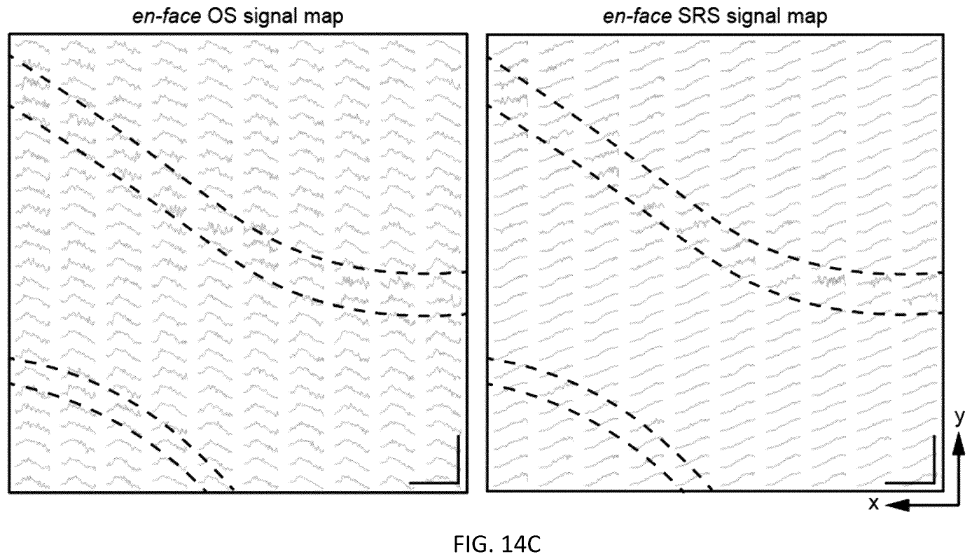

To account for inter-subject differences, phase traces from five rats were combined for feature extraction and subsequent unsupervised clustering. Pre-processing and PCA were conducted on the phase traces to extract their temporal features. A three-dimensional (3D) spatiotemporal feature space was then constructed using the distance to BrM (depth) as the spatial feature and the top two principal components (PCs) as the temporal features (see FIG. 14A). Outliers in low-density regions were removed using a distance-based algorithm (gray dots in FIG. 14A).

To differentiate between two distinct signatures (see FIG. 10D), an agglomerative hierarchical clustering algorithm was employed based on Ward's criterion. Thresholding the dendrogram along the solid black line in FIG. 11B grouped the remaining phase traces (corresponding to the remaining dots in FIG. 11A) into three clusters in the spatiotemporal feature space (FIG. 11C), where a transition band (dots with middle level of shade of grey shown in FIG. 11C) facilitates a better separation between two distinct dynamics signatures. Representative signals (shown in FIG. 11D) were obtained by averaging the individual phase traces within the corresponding cluster and converting them into OPL changes (ΔOPL). The first type of signal (Type-I) exhibited a rapid increase, peaking around 0.5 seconds, followed by a gradual decrease and a negative overshoot after 2.5 seconds (FIG. 11D). This Type-I signal matches the optoretinography signals from the photoreceptor outer segments in the literature, which were published at shorter time ranges. The second type of signal (Type-II) is characterized by a much slower rise, and its peak is not reached within the 3.5-second time range plotted in FIG. 11D.

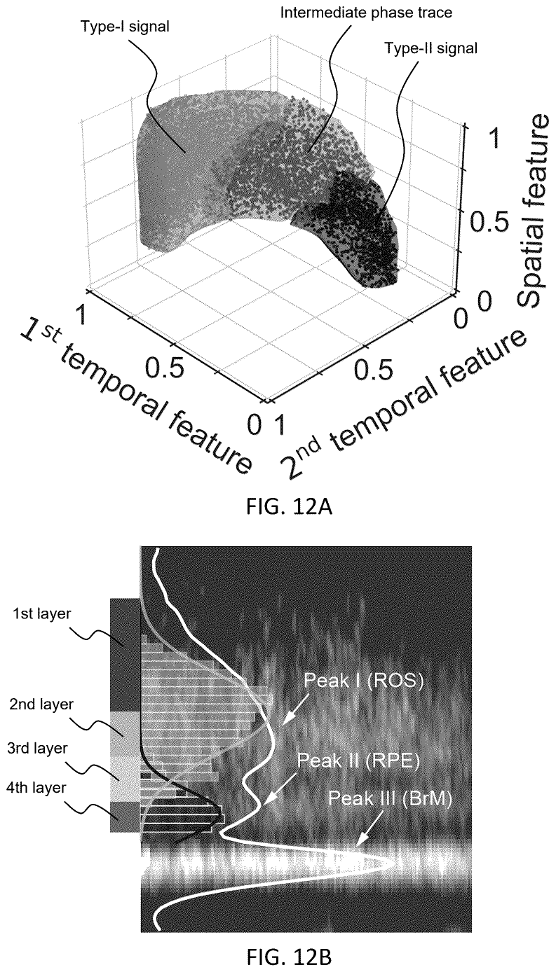

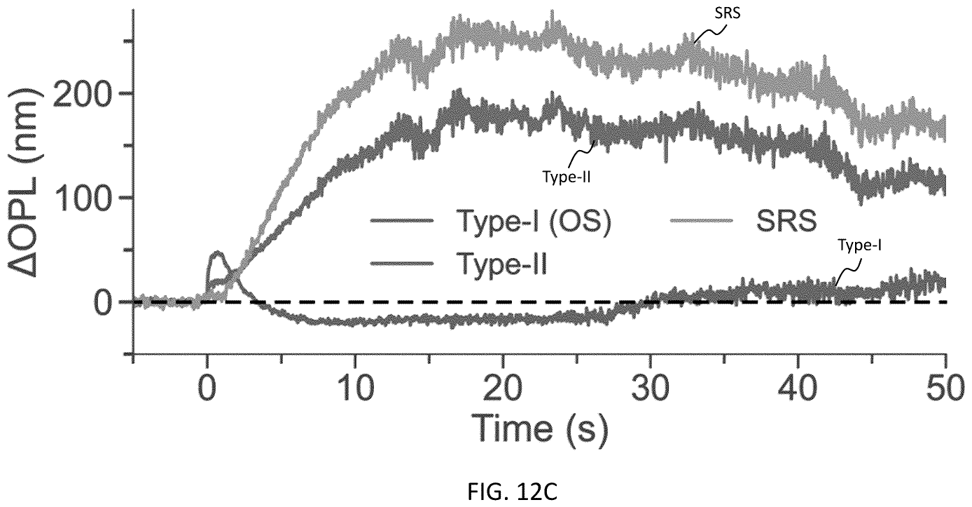

Subsequently, an SVM was trained using the labels obtained from unsupervised clustering in FIG. 11C to set boundaries for Type-I and Type-II signals in the spatiotemporal feature space (see FIG. 12A). FIGS. 12A to 12C illustrate the classification of new phase traces and the validation of their origins, according to various example embodiments of the present invention. In particular, FIG. 12A shows the trained SVM decision boundaries for the Type-I signal and the Type-II signal. Dots represent the phase traces extracted from a new dataset, preprocessed and projected onto the same 3D spatiotemporal feature space. They were classified into Type-I signals, Type-II signals, intermediate phase traces and outliers (not shown). FIG. 12B shows an enlarged view of the dashed box in FIG. 10B, with contrast adjustment to enhance the RPE visibility. Histogram of Type-I and Type-II signals, fitted by Gaussian functions (solid lines), shows their depth distribution on top of the averaged structural image captured by vis-OCT, with a white line depicting its intensity profile. The bars on the left of FIG. 12B correspond to the depth range from which the signals in FIG. 10D were extracted. FIG. 12C depicts plots of Type-I, Type-II and SRS signals from an extended (55 seconds) recording.

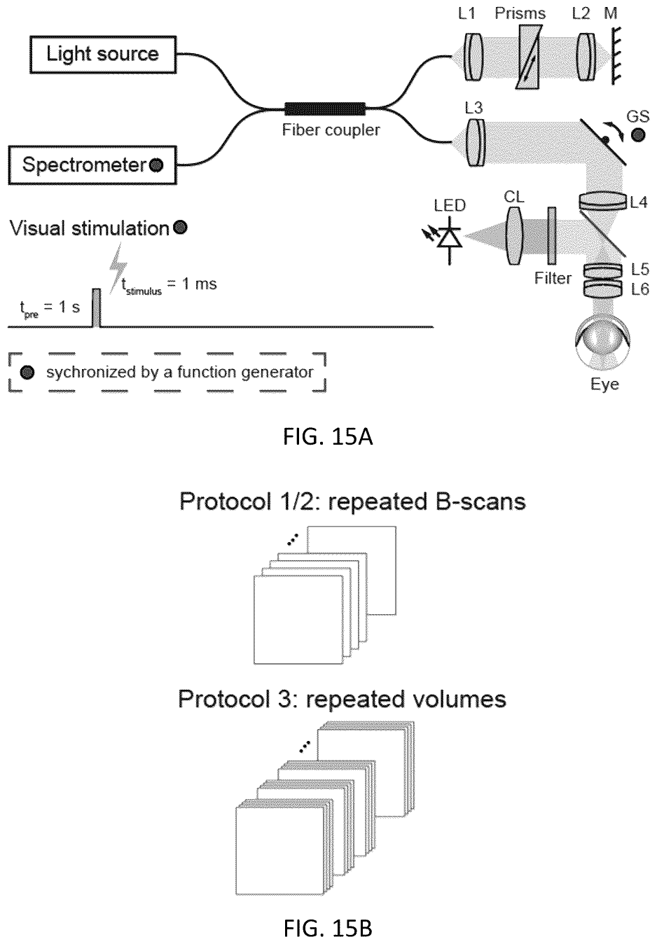

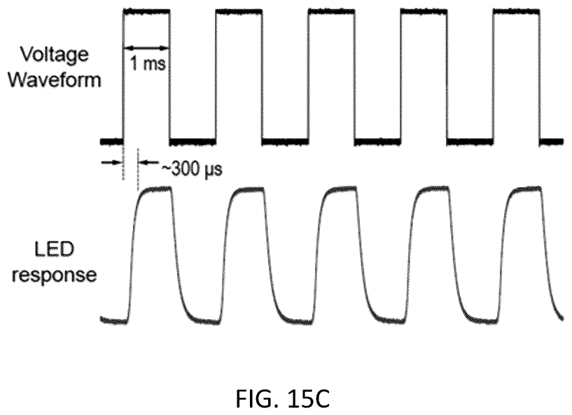

Accordingly, using the pre-trained SVM, various example embodiments successfully extracted Type-I and Type-II signals from new OCT image datasets. Type-I signals were located more anteriorly than Type-II signals, exhibiting a distribution close to normal (grey curve in FIG. 12B). Type-II signals were localized anterior to BrM, fitting a Gaussian function (black curve in FIG. 12B). The locations of the peaks differed significantly (*P<0.05, t-test). The location of Type-I signals corresponded to the intensity profile of OS, while Type-II signals corresponded to location of RPE, as measured by vis-OCT (yellow line in FIG. 15B). This observation suggested that the Type-I and Type-II signals correspond to the dynamics of the OS and RPE relative to IS/OS, respectively. The phase traces were interpolated and clustered from an extended (55 seconds) recording using the SVM. The Type-I signal peaked within one second, underwent a negative undershoot peaking at 10 seconds, and returned to baseline within 30 seconds (see FIG. 12C). By incorporating the Type-II signal with the dynamics between IS/OS and ELM, the dynamics of SRS (from ELM to RPE) was obtained. The SRS signal (SRS trace denoted in FIG. 12C) increased more slowly, peaking at about 250 nm around 20 seconds, followed by an even slower recovery. In the subsequent quantitative analyses, OS and SRS dynamics will be focus on.

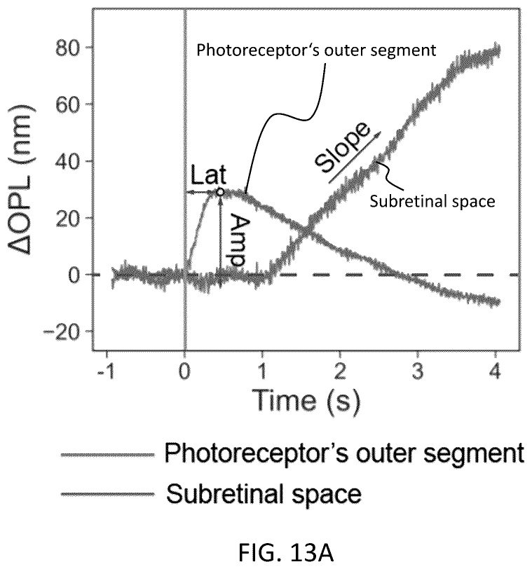

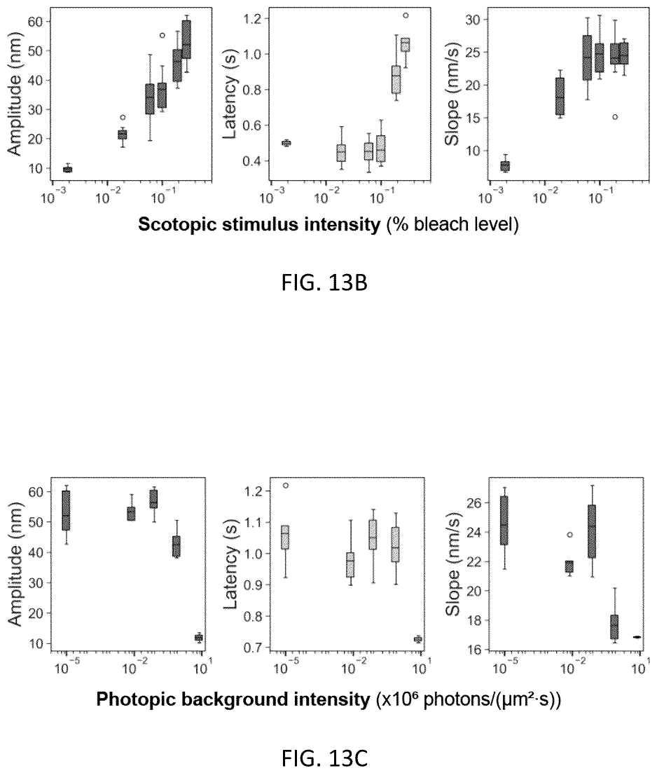

The signal dependence on stimulus parameters will now be described according to various example embodiments of the present invention. FIGS. 13A to 13C illustrate the responses of the outer segment (OS) and subretinal space (SRS) in different conditions. In particular, FIG. 13A shows representative traces of the light-evoked responses in photoreceptor OS and in SRS. FIG. 13B shows amplitude and latency of the OS response, and slope of the SRS expansion as a function of stimulus intensity on scotopic background. FIG. 13C shows plots of photopic background intensity. For box plots, horizontal bar: mean value, box edges: 25 and 75 percentiles, whiskers: 1.5× standard deviations (SDs).

In particular, the signals in scotopic and photopic conditions were quantified by plotting the amplitude and latency of the peak of OS expansion and slope (expansion rate) from the SRS response (see FIG. 13A). In scotopic conditions (see FIG. 13B), the peak amplitude of the OS signal increased logarithmically with the stimulus strengths, from ΔOPL of about 10 (1.72) nm [mean (SD)] at a bleach level of 0.002% to about 53 (8.68) nm when the flash intensity increased 140-fold to 0.28%. The peak latencies remained within 440-500 ms at a bleach level<0.1%, but increased to 877 (130) ms and 1063 (108) ms in response to stimuli at 0.19% and 0.28% bleach level, respectively. In contrast, the slope of the SRS signal increased rapidly with the stimulus intensity up to 0.06% bleach level, and stabilized at the level of about 25 nm/s after that. In photopic conditions (FIG. 13C), the retina was pre-illuminated (500 nm) for 5 minutes and the flash was at 0.28% bleach level. Background illuminance below 0.078×10⁶ photons/(μm²·s) did not reduce the OS response, but it decreased 5-fold at background of 7.8×10⁶ photons/(μm²·s). The OS peak latency was not altered with an even stronger background—up to 0.78×10⁶ photons/(μm²·s), but it dropped by about 30% at 7.8×10⁶ photons/(μm²·s). Similarly, SRS expansion rate was affected by a background illuminance only above 0.078×10⁶ photons/(μm²·s), which reduced the slope by about 30% to 17 nm/s.

FIGS. 14A to 14C show the representative en-face functional maps of outer segment (OS) and subretinal space (SRS) signals. In particular, FIG. 14A shows the volumetric scan covering a 12° field of view, with the structural contrast. FIG. 14B shows the OS and SRS signals at selected time points. Each grid represents the average response from a 0.48°×0.48° (x×y) area. Locations blocked by large blood vessels are outlined by dashed lines. FIG. 14C shows spatiotemporal evolution of the OS and SRS signals over the entire FOV. Each curve presents the average response from a 1.2°×0.48° (x×y) area (scale bar: 200 μm).

In particular, using repeated volumetric scans, the SRS and OS dynamics were mapped, analogous to multifocal ERG, but with a single flash. FIG. 14A displays an OCT volume with a 12° FOV, with the structural contrast. The spatiotemporal distribution of SRS and OS signals was obtained at a 0.10% bleach level. The temporal resolution was 8 Hz, and the total recording time was 5 seconds with 1 second baseline. FIG. 14B shows the OS and SRS signals at specific time points. FIG. 14C maps the spatial distribution of the phase traces, with each grid representing the average response from a 1.2°×0.48° area. In general, the OS and SRS signal maps show high-fidelity detection over the entire FOV, and high signal variance was observed underneath two large blood vessels, outlined by dashed lines in FIGS. 14B and 14C.

Signals from SRS and OS, recorded over a wide field in response to a single flash, offer a convenient approach to diagnostic mapping, in contrast to the slow and low-resolution multifocal ERG. This technique expands the practical applicability of optoretinography to studies of not only photoreceptors but also the RPE's control of water dynamics in subretinal space in health and disease, thus capable of being a more complete replacement for ERG.

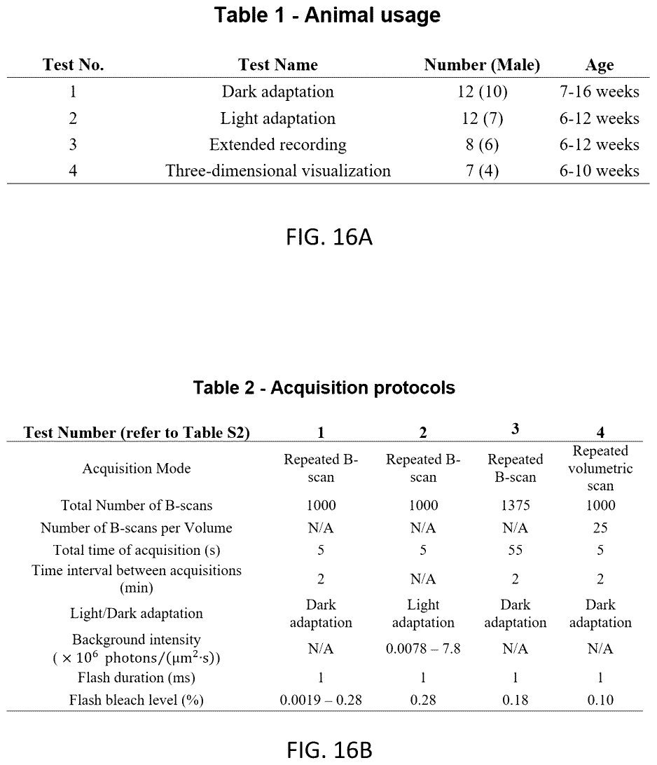

An example system setup according to various example embodiments of the present invention will now be described, as well as example stimulus scheme and acquisition protocols, with reference to FIGS. 15A to 15C. FIG. 15A depicts a spectral-domain OCT (ultrahigh axial resolution) was used to image the posterior segment of rat eyes. A line-scan camera interfaced with the spectrometer was used to acquire the interference fringes. A function generator synchronized the line-scan camera acquisition, galvo scanner rotation, and flash timing. In FIG. 15A, L1-L6 denote doublet lenses. CL denotes condenser lens, GS denotes galvo scanner, and the filter spectral window is 500±5 nm. FIG. 15B shows three acquisition protocols which were used, namely, repeated B-scans (Protocol 1: 1000 A-scans per B-scan, 200 B-scans per second, Protocol 2: 1000 A-scans per B-scan, 25 B-scans per second) and repeated volumes (Protocol 3: 1000 A-scans per B-scan, 25 B-scans per volume, 8 volumes per second). The details of acquisition and stimulation protocols are presented in Table 2 in FIG. 16B. As an illustrative example described hereinbefore, FIG. 6C shows the time-elapsed intensity (upper) and phase (lower) M-scans without correction, when no light stimulus was delivered to the retina (scale bar: 500 ms (horizontal), 100 μm (vertical)), the subpixel-level bulk motion estimated by locating the peak of the upsampled cross-correlation map between repeated B-scans (scale bar: 500 ms (horizontal), 5 μm (vertical)) and the corresponding time-elapsed intensity (upper) and phase (lower) M-scans after the phase-restoring subpixel motion correction (scale bar: 500 ms (horizontal), 100 μm (vertical)).

Optoretinography imaging experiments were performed using a custom-built spectral-domain OCT shown in FIG. 15A. The NIR-OCT system operated with a broadband superluminescent diode (cBLMD-T-850-HP-I, λ_{c}=840 nm, Δλ=146 nm, Superlum, Ireland), providing an axial resolution of 2.0 μm in tissue. A spectrometer interfaced with a line-scan camera (2048 pixel, 250,000 Hz Cobra-800, Octoplus, E2V) acquired the spectral interference fringes at an A-scan speed of 250 kHz, corresponding to an image depth of 1.07 mm in air. For posterior segment imaging in rodent eyes, a lens-based afocal telescope conjugated the axis of a galvo scanner to the pupil. To reduce the incident beam size and increase the scan angle, the telescope's magnification was 0.17 (scan lens: 80 mm focal length; ocular lens: 30 mm+25 mm focal length). The theoretical diffraction-limited lateral resolution was 7.2 μm with a standard rat eye model. A function generator (PCIe-6363, National Instruments, USA) synchronized the camera acquisition (both A-scan and B-scan acquisitions), galvanometer scanning, and visual stimulation.

For visual stimulation, a LED (MBB1L3, Thorlabs, USA) was collimated by an aspheric condenser lens, and a narrow bandpass filter (500±5 nm, #65-694, Edmund Optics, Singapore) reshaped the spectrum to optimize the sensitivity to rhodopsin (32). LED's response time is about 300 s, equivalent to 6% of the B-scan frame acquisition time (5 ms), and LED's response waveform to trigger is shown in FIG. 15C. A 43.4°, Maxwellian illuminance was projected in the posterior eye to cover an area of approximately 6.75 mm². The power and duration of the light stimulus were converted to the bleach percentages of rhodopsin.

A visible-light optical coherence tomography (vis-OCT) at higher axial resolution of 1.1 μm in tissue was constructed to resolve the ultra-fine laminated structure of the outer retina. Briefly, the system used a supercontinuum laser (SuperK Extreme, NKT Photonics, Denmark) with a spectrum truncated between 435 and 650 nm. Vis-OCT adopted a similar optical design as the NIR system, albeit all the components work in the visible spectral range. A spectrometer (Cobra VIS, Wasatch, USA) interfaced with a line-scan camera acquired the spectra of the interference fringes at an A-scan speed of 50 kHz. Volumetric raster scans (500 A-scans×500 B-scans) were collected within a 12°×1.2° (1.14 mm×0.11 mm) rectangle field of view. Sequential B-scans were aligned and then averaged into a single frame.

The animal experiment protocol will now be described. These experiments were conducted in accordance with guidelines and approvals from Institutional Animal Care and Use Committee (IACUC), SingHealth (2020/SHS/1574). Brown Norway rats (N=39) were used with details present in Table 1 in FIG. 16A). Animals were sedated using a ketamine/xylazine combination to better maintain retinal functional responses compared to other commonly used anesthetics, such as isoflurane and urethane, while minimizing eye motion. Vital signals, including heart and respiration rates, were monitored throughout the imaging sessions. Animals were placed in a prone position with their head restrained stereotaxically. Two mydriasis drops, 1% Tropicamide (Alcon, Geneva, Switzerland) and 2.5% Phenylephrine (Alcon, Geneva, Switzerland), were administrated onto the cornea before the imaging. The cornea was moisturized by a balanced salt solution frequently throughout the imaging sessions.

M-opsin and rhodopsin have similar sensitivity spectra (peaking at approximately 500 nm), while S-opsin's sensitivity peaks at 350 nm, with minimal overlap with rhodopsin sensitivity spectrum. Since M-opsin co-expression ratio decreases from ventral to dorsal, the scanning region was limited to the dorsal area to minimize the influence of M-opsin in cones.

Details of the acquisition and stimulation protocols are presented in Table 2 in FIG. 16B. Three scanning protocols were used in experiments conducted. In a first protocol, 1000 A-scans per B-scan and 200 B-scans per second were acquired, with a total acquisition time of 5 seconds. In a second protocol, the B-scan time interval was increased to 40 ms and 1375 B-scans were acquired over a period of 55 seconds. In a third protocol, 40 repeated volumes (25 B-scans per volume) were acquired, corresponding to 8 volumetric scans per second for a period of 5 seconds. Flash intensity, dark/light adaptation and inter-flash time interval were varied in different experiments.

The automated extraction of the outer retina dynamics from speckle patterns will now be described according to various example embodiments of the present invention.

First, the preprocessing of OCT images and phase traces was performed. In this regard, the raw interference fringe first underwent standard OCT post-processing steps, including spectral calibration, k-linearity, dispersion compensation, and discrete Fourier transform (DFT), which resulted in complex-valued OCT images.

Phase-sensitive OCT is very susceptible to bulk tissue motion, which results in apparent image distortions in the time-elapsed intensity M-scan and degrades the phase stability (see left portion of FIG. 6C). To correct the motion-induced phase error, subpixel-level translational displacements were estimated between repeated B-scans using the single-step DFT algorithm (see center portion of FIG. 6C). The complex-valued OCT images was registered using a phase-restoring subpixel motion correction algorithm, where the lateral and axial displacements were corrected by multiplying the corresponding exponential terms in the spatial frequency domain and the spectrum domain, respectively. After image registration (see right portion of FIG. 6C), excellent motion stability was achieved at photoreceptor layers (white arrows), while periodical oscillations from the vascular pulsation (grey arrows) can be observed in the choroid.

Light-evoked dynamics of the outer retina was then extracted by computing the temporal phase difference between the pixel pairs from the outer retinal bands. Several hyperreflective bands, including ELM, IS/OS, BrM, and a thick speckling layer that contains photoreceptors' OS and RPE cells, were automatically segmented using graph theory and dynamic programming. In self-referencing measurements, for each pixel in a target layer, its temporal phase change was computed with respect to a reference layer.

Temporal filtering was also conducted to remove unwanted signal frequencies. The extracted signals were first processed by bandstop filters to minimize residual artifacts induced by heartbeat and breathing. A low-pass filter with a cut-off frequency of 10 Hz was subsequently used to filter out high-frequency oscillations. After filtering, both ends of the phase traces exhibited high variance due to edge effects, so they were removed from the data analysis afterwards.

Regarding the construction of spatiotemporal feature space according to various example embodiments, the PCA was used to compress high-dimensional phase traces into a lower-dimensional feature space to facilitate the analysis. Phase traces with a pre-stimulus SD larger than 60 mrad were excluded, mostly from pixels with low SNR or underneath blood vessels. Subsequently, each phase trace was normalized by subtracting its mean value and dividing by its SD. To account for inter-subject differences, normalized traces extracted from five rats were combined to calculate PC coefficients. A 3D spatiotemporal feature space was constructed, whereby the top two PCs capturing a total variance of 73.3% were selected as temporal features and its axial distance to the BrM was selected as the spatial feature.

To avoid undesirable elongated clusters in subsequent unsupervised clustering analysis, a distance-based outlier detection method was used to preclude outliers distributed in low-density regions in the spatiotemporal feature space. For each data point in the spatiotemporal feature space, the minimum radius of a sphere that is centered at that point and can cover 2% of the remaining data points was determined. This radius reflected the local distribution density around each data point in the spatiotemporal feature space. Then, a given point would be labeled as an outlier if its corresponding radius was larger than Q₃+(Q₃−Q₁)/5, where Q₁ and Q₃ are the first and third quartiles of the calculated radii.

Regarding the unsupervised clustering with a hierarchical clustering algorithm according to various example embodiments, an agglomerative hierarchical clustering algorithm under the Ward criterion was used to group individual points in the established spatiotemporal feature space. The Euclidean distance of each pair of points was computed and similar subclusters were then iteratively merged into larger clusters. Under the guidance of the Ward method or minimum variance method, each merger guaranteed a minimum increase of total within-cluster variance.

Regarding the training of a support vector model in the established spatiotemporal feature space according to various example embodiments, a SVM was trained in the spatiotemporal feature space with the previously obtained labels (labelled feature points), including outlier, intermediate phase traces, Type-I signal, and Type-II signal, to obtain their decision boundaries. The input features were standardized before training, the Gaussian kernel was selected, and automatic hyperparameters optimization was turned on. Evaluated with the 10-fold cross-validation strategy, the trained SVM achieved a classification accuracy of 99.2%. Accordingly, the SVM is useful for processing a new dataset using the same criterion. In this regard, phase traces extracted from the new OCT image dataset can be preprocessed and projected onto the same spatiotemporal feature space. Then, for example, Type-I and Type-II signals can be automatically extracted based on the classification results from the trained SVM.

Regarding the processing of datasets with lower temporal resolution according to various example embodiments, extended recording (protocol 2) and volumetric scans (protocol 3) had an 8-fold and 25-fold lower temporal sampling rate than repeated B-scans (protocol 1), respectively. To cluster these phase traces in the same spatiotemporal feature space, they were first linearly interpolated into 5 ms time intervals and smoothed using a Gaussian filter. Other procedures were similar to those used for processing the repeated B-scans datasets.

A visible-light, spectral-domain optical coherence tomography (vis-OCT) was constructed according to various example embodiments using a supercontinuum laser covering 400-2300 nm spectrum (SuperK Extreme, NKT Photonics, Denmark) as the light source. A spectrum ranging from 435 nm to 650 nm with a full-width at half-maximum bandwidth of 120 nm was selected by a spectral splitter (SuperK SPLIT, NKT Photonics, Denmark) and a shortpass filter (#47-290, Edmund Optics, USA), and further reshaped by a bandpass filter (#16-362, Edmund Optics, USA). In the sample arm, a customized achromatizing triplet lens was placed between the reflective collimator and the galvo scanner (Saturn5, ScannerMax, USA) to reduce the chromatic aberration of the rat eye. A lens-based afocal telescope conjugated the center of the galvo scanner pair to the pupil with a magnification of 0.18 (scan lens: 75 mm focal length; ocular lens: 30 mm+25 mm focal length) to reduce the incident beam diameter to 310 μm at the pupil.

Accordingly, various example embodiments demonstrated an automated, unbiased optoretinography for rodents with a non-AO, point-scan, SD-OCT. For example, two types of signals were automatically identified from speckled images using an agglomerative hierarchical clustering analysis conducted in the PC space. Accordingly, a SVM model was trained to explore the decision boundaries of each type of signal in the established PC space, facilitating the classification of new phase traces extracted from, for example, new animals. For example, various example embodiments can simultaneously study outer retinal dynamics from various tissue types, and generate geographic maps of tissue-specific dynamics, analogous to multifocal electroretinogram, with only a single brief flash. Moreover, various example embodiments developed a general protocol that is applicable to conventional OCT systems in animal labs and clinics without demanding hardware or human intervention, with a remarkable potential in hunting for tissue-specific dynamics without prior knowledge.

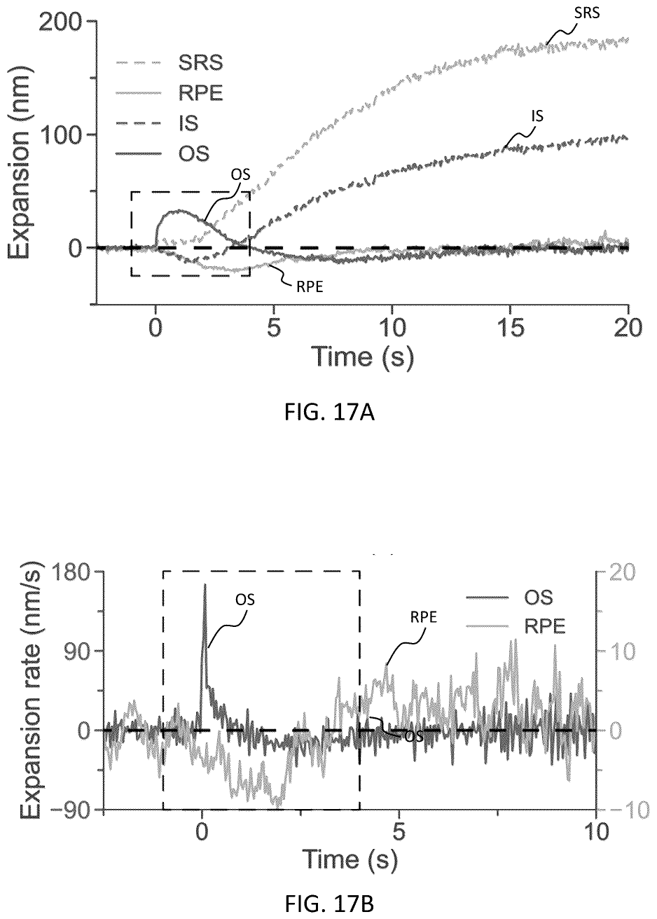

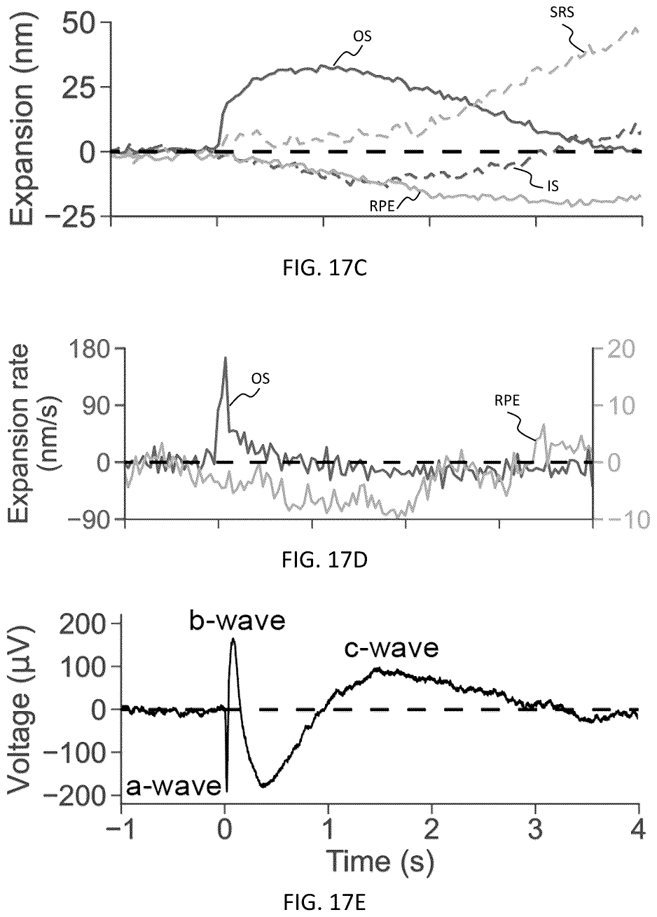

According to various example embodiments, after disambiguation of various signals in the outer retina, dynamics of its cellular layers can be described independently. Time derivative of the tissue deformation, i.e., its expansion rate, is very informative since it may reveal the water influx rate. FIGS. 17A to 17E show the deformation of cellular layers over time and comparison of optoretinography with ERG. FIG. 17A shows the dynamics of the deformations of the rod outer segment (OS), inner segment (IS), retinal pigment epithelium (RPE), and subretinal space (SRS, from the BrM to ELM) after a 1 ms green stimulus at 0.26% bleach level. FIG. 17B shows the expansion rate of the OS and RPE averaged across 5 measurements. FIGS. 17C and 17D show zoom-in view of the dashed boxed data in FIGS. 17A and 17B, respectively. FIG. 17E shows an example electroretinography (ERG) trace in response to a white flash, where the a, b and c waves are labeled. OPL change was converted into physical deformation with a refractive index of 1.41.

As shown in FIGS. 17A to 17E, expansion rate of OS reaches its maximum of about 165 nm/s within 0.1 second after the stimulus and drops back to zero during the next 1 second. RPE, on the other hand, reaches the maximum contraction rate of about −9 nm/s at 2 seconds after the stimulus, leading to the maximum contraction of approximately 20 nm at 3 seconds, and slowly recovering afterwards. Expansion of SRS begins about 1.5 seconds after the stimulus, reaching a rate of 15 nm/s in the following 10 seconds, after which the expansion rate gradually decreases. SRS expansion may originate from water transport across the RPE in response to a decrease in K⁺ and an increase in Na⁺ concentrations due to phototransduction in photoreceptors, as observed earlier using other methods. The inner segment is compressed by about 10 nm during 1.5 seconds, after which it follows the dynamics of SRS, albeit at a lower amplitude (100 nm expansion at maximum).

It is interesting to relate the optoretinography signals to ERG previously recorded in the same species—pigmented rats, in response to a white flash, shown in FIG. 17E. The a-wave in ERG—the photoreceptors' response to light, begins right after the flash (within 10 ms), corresponding to the beginning of the OS expansion in optoretinography. The continuation of the a-wave is obscured by the b-wave, which starts a few tens of milliseconds later and corresponds to the electric current generated by the second-order retinal neurons. C-wave in ERG takes over after about 0.5 seconds and reaches its maximum at around 1.5 seconds. It corresponds to a potassium current through RPE, induced by the photoreceptors' response to light. The RPE contraction in optoretinography, which reaches its maximum rate between 1 and 2 seconds, overlaps with the peak of c-wave (see FIGS. 17D and 17E). This new optical signature opens the window into physiology of the photoreceptor-RPE interactions and the interphotoreceptor matrix. Water transport is limited by the membrane permeability and hence its dynamics is much slower than that of electric current. Using a model of the OS elongation due to osmotic imbalance during phototransduction, the membrane permeability coefficient was estimated based on the OS expansion rate. The water permeability coefficient of the rod OS membrane was found to be 5.9×10⁻³ cm·s⁻¹, not too far from 2.6×10⁻³ cm·s⁻¹ measured earlier in vitro.

Intriguingly, the hyperpolarized RPE layer is getting compressed in this process, unlike swelling of the hyperpolarized outer segments driven by osmotic pressure changes in response to released osmolytes during phototransduction.

The responses of rods and cones to light vary in many aspects, including sensitivity, time constant and light adaptation. These differences may be attributed to different expressions of isoforms involved in the phototransduction cascade and distinct structural organizations of plasma membranes in rods and cones. Until now, optoretinography was observed mainly in cone photoreceptors, since detecting rod signals in human subjects is more challenging because rods are generally smaller and densely packed around cones. In rats, however, 97% of photoreceptors are rods, and hence the OS signals are likely dominated by the rod outer segments (ROS). Adaptive optics (AO) enables imaging of single rods in peripheral human retina, and one AO-OCT study reported that a flash bleaching 0.05% rhodopsin resulted in rod OS elongation of about 60 nm, while using the same flash bleaching 0.2% opsin did not result in a detectable cone OS elongation. These results agree with our observations: 0.06% bleach caused OS elongation by 20-30 nm in rats.

In a previous study using conventional intensity-based OCT, Zhang et al., “In vivo optophysiology reveals that G-protein activation triggers osmotic swelling and increased light scattering of rod photoreceptors”, Proceedings of the National Academy of Sciences, U.S.A., April 2017 observed a slow (peak latency of 10-100 seconds, depending on the bleach level) increase in distance between IS/OS and BrM in response to light, and attributed it to elongation of the OS. Notably, this slow signal resembles our SRS expansion, while the actual OS signal is much faster to rise and fall in the observation herein and in previous reports.

Another interesting finding is the undershoot of the OS signal, with its tens of seconds-long recovery (FIG. 17A), which was not reported in previous studies due to shorter observation time. It may be related to water transport from the OS to SRS when the osmolytes (Gat, Gβ₁γ₁) rebind to the cell membrane during deactivation of phototransduction. Restoration of the OS osmotic pressure during expansion of the SRS may be accompanied by its slight (20 nm) compression, which recovers later along with the other cellular structures in the outer retina.

Expansion of the SRS, closely related to phototransduction and water transport via RPE, is larger than that of the OS. Such expansion may be used as a more sensitive measure of retinal physiology, providing additional diagnostic insights for various diseases involving the outer retina, optoretinography conveniently combines structural and functional retinal imaging in the same machine.

Accordingly, an automated, unbiased approach to extracting wide-field, depth-resolved optoretinography signals has been disclosed according to various example embodiments, which reveal comprehensive nanoscopic dynamics of the outer retina in preclinical models. This method can be widely applied to study the tissue-specific dynamics in various animal models. For example, the detected outer retinal dynamics can serve as a new piece of evidence that helps improve the understanding of the phototransduction cascades. At the same time, it holds remarkable potential in the applications of investigating disease models and facilitating clinical translations.

For example, the method of performing optoretinography using OCT according to various example embodiments is capable of significantly promoting noninvasive imaging of nanometer-scale movement or cellular deformation in clinical applications on all sorts of FD-OCT systems, including point-scan, line-scan, and full-field systems. While OCT has been widely used for structural imaging in ophthalmology, the method according to various example embodiments improves the accuracy of imaging cellular dynamics which may lead to new market space in functional diagnosis, particularly for the emerging optoretinogram that measures the physiological response of the retina non-invasively and all-optically. For example, since the method according to various example embodiments allows accurate correction of the phase components disturbed by the inevitable bulk motions in live tissue without additional hardware, it can be easily applied to any clinical OCT system that utilizes phase components for imaging, hence, resulting in rapid commercialization to a large scale.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A method of training a machine learning model for classifying a temporal phase signal obtained from optical coherence tomography (OCT) images produced for optoretinography using at least one processor, the method comprising:
obtaining a series of OCT images produced by a phase-sensitive OCT system for optoretinography with respect to an outer retina, including a stimulus sub-series of OCT images produced in response to a visual stimulus to the outer retina;
determining, for each OCT image of the series of OCT images and for each of a plurality of individual pixels in a target layer of the OCT image, a phase signal of the individual pixel in the target layer of the OCT image with respect to a reference layer of the OCT image to obtain, for each series of corresponding individual pixels of a plurality of series of corresponding individual pixels in the target layer across the series of OCT images, a temporal phase signal of the series of corresponding individual pixels, resulting in a set of temporal phase signals for the plurality of series of corresponding individual pixels, the target layer comprising a plurality of outer retinal bands;
projecting a plurality of temporal phase signals of the set of temporal phase signals to a plurality of feature points in a feature space;
labelling each of the plurality of feature points in the feature space; and
training the machine learning model based on the plurality of labelled feature points in the feature space.

2. The method according to claim 1, wherein said determining the phase signal of the individual pixel in the target layer of the OCT image comprises correcting a systematic phase drift of a complex-valued OCT signal of the individual pixel based on self-referencing to an individual pixel of the reference layer of the OCT image to obtain a first complex-valued OCT signal of the individual pixel.

3. The method according to claim 2, wherein said correcting the systematic phase drift of the complex-valued OCT signal of the individual pixel comprises multiplying the complex-valued OCT signal of the individual pixel with a complex conjugate of the complex-valued OCT signal of the individual pixel of the reference layer of the OCT image to obtain the first complex-valued OCT signal of the individual pixel.

4. The method according to claim 3, wherein, for each OCT image of the stimulus sub-series of OCT images, said determining the phase signal of the individual pixel in the target layer of the OCT image further comprises correcting a phase offset of the first complex-valued OCT signal of the individual pixel based on a pre-stimulus sub-series of the series of OCT images produced prior to the visual stimulus to the outer retina to obtain a second complex-valued OCT signal of the individual pixel.

5. The method according to claim 4, wherein said correcting the phase offset of the first complex-valued OCT signal of the individual pixel comprises:
determining an average of the first complex-valued OCT signals of corresponding individual pixels of the pre-stimulus sub-series of OCT images corresponding to the individual pixel of the OCT image to obtain an averaged pre-stimulus complex-valued OCT signal; and
multiplying the first complex-valued OCT signal of the individual pixel with the averaged pre-stimulus complex-valued OCT signal to obtain the second complex-valued OCT signal of the individual pixel.

6. (canceled)

7. The method according to claim 5, wherein
the phase signal of the individual pixel in the target layer of the OCT image is determined with respect to a reference pixel region of the reference layer of the OCT image, the reference pixel region comprising a plurality of individual pixels,
for said correcting the systematic phase drift of the complex-valued OCT signal of the individual pixel, said multiplying the complex-valued OCT signal of the individual pixel comprises multiplying the complex-valued OCT signal of the individual pixel respectively with complex conjugates of the complex-valued OCT signals of the plurality of individual pixels of the reference pixel region of the reference layer of the OCT image to obtain a set of first complex-valued OCT signals of the individual pixel, and
for said correcting the phase offset of the complex-valued OCT signal of the individual pixel,
said determining the average of the first complex-value OCT signals of corresponding individual pixels comprises determining an average of the sets of first complex-valued OCT signals of corresponding individual pixels of the pre-stimulus sub-series of OCT images corresponding to the individual pixel of the OCT image to obtain an averaged set of pre-stimulus complex-valued OCT signals; and
said multiplying the first complex-valued OCT signal of the individual pixel comprises multiplying the set of first complex-valued OCT signals of the individual pixel with the averaged set of pre-stimulus complex-valued OCT signals to obtain a set of second complex-valued OCT signals of the individual pixel.

8. The method according to claim 7, wherein for each OCT image of the stimulus sub-series of OCT images, said determining the phase signal of the individual pixel in the target layer of the OCT image further comprises:
determining an average of the set of second complex-valued OCT signals of the individual pixel to obtain an averaged complex-valued OCT signal of the individual pixel; and
determining the phase signal of the individual pixel in the target layer of the OCT image based on the averaged complex-valued OCT signal of the individual pixel.

9. (canceled)

10. The method according to claim 1, wherein said labelling each of the plurality of feature points in the feature space comprises:
grouping the plurality of feature points into a plurality of clusters in the feature space; and
labelling, for each of the plurality of clusters, feature points belonging to the cluster with a label assigned to the cluster.

11. (canceled)

12. The method according to claim 10, wherein the plurality of clusters has a plurality of different labels assigned thereto, respectively, the plurality of different labels comprising a plurality of different outer retinal band labels corresponding to the plurality of outer retinal bands of the target layer.

13. The method according to claim 1, wherein the reference layer corresponds to the inner segment/outer segment (IS/OS) junction, and the plurality of outer retinal bands comprises two or more outer retinal bands respectively corresponding to two or more of a photoreceptor outer segment, a microvilli, a retinal pigment epithelium (RPE) and a Bruch's membrane.

14. (canceled)

15. A method of classifying a temporal phase signal obtained from optical coherence tomography (OCT) images produced for optoretinography using the trained machine learning model trained according to claim 1, the method comprising:
obtaining a series of OCT images produced by a phase-sensitive OCT system for optoretinography with respect to an outer retina, including a stimulus sub-series of OCT images produced in response to a visual stimulus to the outer retina;
determining, for each individual pixel of a series of corresponding individual pixels in a target layer across the series of OCT images, a phase signal of the individual pixel in the target layer of the OCT image with respect to a reference layer of the OCT image to obtain a temporal phase signal of the series of corresponding individual pixels, the target layer comprising a plurality of outer retinal bands;
projecting the temporal phase signal of the series of corresponding individual pixels to a feature point in a feature space; and
classifying the temporal phase signal using the trained machine learning model based on the feature point which the temporal phase signal has been projected to.

16. The method according to claim 15, wherein said determining the phase signal of the individual pixel in the target layer of the OCT image comprises correcting a systematic phase drift of a complex-valued OCT signal of the individual pixel based on self-referencing to an individual pixel of the reference layer of the OCT image to obtain a first complex-valued OCT signal of the individual pixel.

17. The method according to claim 16, wherein said correcting the systematic phase drift of the complex-valued OCT signal of the individual pixel comprises multiplying the complex-valued OCT signal of the individual pixel with a complex conjugate of the complex-valued OCT signal of the individual pixel of the reference layer of the OCT image to obtain the first complex-valued OCT signal of the individual pixel.

18. The method according to claim 17, wherein, for each OCT image of the stimulus sub-series of OCT images, said determining the phase signal of the individual pixel in the target layer of the OCT image further comprises correcting a phase offset of the first complex-valued OCT signal of the individual pixel based on a pre-stimulus sub-series of the series of OCT images produced prior to the visual stimulus to the outer retina to obtain a second complex-valued OCT signal of the individual pixel.

19. The method according to claim 18, wherein said correcting the phase offset of the first complex-valued OCT signal of the individual pixel comprises:
determining an average of the first complex-valued OCT signals of corresponding individual pixels of the pre-stimulus sub-series of OCT images corresponding to the individual pixel of the OCT image to obtain an averaged pre-stimulus complex-valued OCT signal; and
multiplying the first complex-valued OCT signal of the individual pixel with the averaged pre-stimulus complex-valued OCT signal to obtain the second complex-valued OCT signal of the individual pixel.

20. (canceled)

21. The method according to claim 19, wherein
the phase signal of the individual pixel in the target layer of the OCT image is determined with respect to a reference pixel region of the reference layer of the OCT image, the reference pixel region comprising a plurality of individual pixels,
for said correcting the systematic phase drift of the complex-valued OCT signal of the individual pixel, said multiplying the complex-valued OCT signal of the individual pixel comprises multiplying the complex-valued OCT signal of the individual pixel respectively with complex conjugates of the complex-valued OCT signals of the plurality of individual pixels of the reference pixel region of the reference layer of the OCT image to obtain a set of first complex-valued OCT signals of the individual pixel, and
for said correcting the phase offset of the complex-valued OCT signal of the individual pixel,
said determining the average of the first complex-value OCT signals of corresponding individual pixels comprises determining an average of the sets of first complex-valued OCT signals of corresponding individual pixels of the pre-stimulus sub-series of OCT images corresponding to the individual pixel of the OCT image to obtain an averaged set of pre-stimulus complex-valued OCT signals; and
said multiplying the first complex-valued OCT signal of the individual pixel comprises multiplying the set of first complex-valued OCT signals of the individual pixel with the averaged set of pre-stimulus complex-valued OCT signals to obtain a set of second complex-valued OCT signals of the individual pixel.

22. The method according to claim 21, wherein for each OCT image of the stimulus sub-series of OCT images, said determining the phase signal of the individual pixel in the target layer of the OCT image further comprises:
determining an average of the set of second complex-valued OCT signals of the individual pixel to obtain an averaged complex-valued OCT signal of the individual pixel; and
determining the phase signal of the individual pixel in the target layer of the OCT image based on the averaged complex-valued OCT signal of the individual pixel.

23. (canceled)

24. The method according to claim 15, wherein the temporal phase signal is classified using the trained machine learning model into one of a plurality of outer retinal tissue types corresponding to a plurality of outer retinal bands of the target layer or an outlier type.

25. The method according to claim 15, wherein the reference layer corresponds to the inner segment/outer segment (IS/OS) junction, and the plurality of outer retinal bands comprises two or more outer retinal bands respectively corresponding to two or more of a photoreceptor outer segment, a microvilli, a retinal pigment epithelium (RPE) and a Bruch's membrane.

26. (canceled)

27. (canceled)

28. A system for classifying a temporal phase signal obtained from optical coherence tomography (OCT) images produced for optoretinography, the system comprising:
at least one memory; and
at least one processor communicatively coupled to the at least one memory and configured to perform the method of classifying a temporal phase signal according to claim 15.
